SEQUENCE LISTING (46 sequences)

SEQ ID NO: 1 - protein, 1049 aa, Homo sapiens
  source 1..1049: note = Wild-type human alpha5 integrin (Uniprot Reference No: P08648)
MGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 2 - protein, 1053 aa, Mus musculus
  source 1..1053: note = Wild-type mouse alpha5 integrin (Uniprot Reference No: P11688)
MGSWTPRSPRSPLHAVLLRWGPRRLPPLLPLLLLLWPPPLQVGGFNLDAEAPAVLSGPPGSLFGFSVEFYRPGRDGVSVLVGAPKANTSQPGVLQGGAVYVCPWGTSPIQCTTIQFDSKGSRILESSLYSAKGEEPVEYKSLQWFGATVRAHGSSILACAPLYSWRTEKDPQNDPVGTCYLSTENFTRILEYAPCRSDFGSAAGQGYCQGGFSAEFTKTGRVVLGGPGSYFWQGQILSATQEQISESYYPEYLINPVQGQLQTRQASSVYDDSYLGYSVAVGEFSGDDTEDFVAGVPKGNLTYGYVTVLNGSDIHSLYNVSGEQMASYFGYAVAATDTNGDGLDDLLVGAPLLMERTADGRPQEVGRVYIYLQRPAGIDPTPTLTLTGQDEFSRFGSSLTPLGDLDQDGYNDVAIGAPFGGEAQQGVVFIFPGGPGGLSTKPSQVLQPLWAAGRTPDFFGSALRGGRDLDGNGYPDLIVGSFGVDKALVYRGRPIISASASLTIFPSMFNPEERSCSLEGNPVSCINLSFCLNASGKHVPNSIGFEVELQLDWQKQKGGVRRALFLTSKQATLTQTLLIQNGAREDCREMKIYLRNESEFRDKLSPIHIALNFSLDPKAPMDSHGLRPVLHYQSKSRIEDKAQILLDCGEDNICVPDLQLDVYGEKKHVYLGDKNALNLTFHAQNLGEGGAYEAELRVTAPLEAEYSGLVRHPGNFSSLSCDYFAVNQSRQLVCDLGNPMKAGTSLWGGLRFTVPHLQDTKKTIQFDFQILSKNLNNSQSNVVSFPLSVEAQAQVSLNGVSKPEAVIFPVSDWNPQDQPQKEEDLGPAVHHVYELINQGPSSISQGVLELSCPQALEGQQLLYVTKVTGLSNCTSNYTPNSQGLELDPETSPHHLQKREAPGRSSTASGTQVLKCPEAKCFRLRCEFGPLHRQESRSLQLHFRVWAKTFLQREYQPFSLQCEAVYEALKMPYQILPRQLPQKKLQVATAVQWTKAEGSNGVPLWIIILAILFGLLLLGLLIYVLYKLGFFKRSLPYGTAMEKAQLKPPATSDA

SEQ ID NO: 3 - protein, 798 aa, Homo sapiens
  source 1..798: note = Wild-type human beta1 integrin (Uniprot Reference No: P05556-1)
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 4 - protein, 798 aa, Mus musculus
  source 1..798: note = Mouse beta1 integrin (Uniprot Reference No: P09055-1)
MNLQLVSWIGLISLICSVFGQTDKNRCLKANAKSCGECIQAGPNCGWCTNTTFLQEGMPTSARCDDLEALKKKGCQPSDIENPRGSQTIKKNKNVTNRSKGMAEKLRPEDITQIQPQQLLLKLRSGEPQKFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTDRGEFFNELVGQQRISGNLDSPEGGFDAIMQVAVCGSLIGWRNVTRLLVFSTDAGFHFAGDGKLGGIVLPNDGQCHLENNVYTMSHYYDYPSIAHLVQKLSENNIQTIFAVTEEFQPVYKELKNLIPKSAVGTLSGNSSNVIQLIIDAYNSLSSEVILENSKLPDGVTINYKSYCKNGVNGTGENGRKCSNISIGDEVQFEISITANKCPNKESETIKIKPLGFTEEVEVVLQFICKCNCQSHGIPASPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCRCRVCECYPNYTGSACDCSLDTGPCLASNGQICNGRGICECGACKCTDPKFQGPTCETCQTCLGVCAEHKECVQCRAFNKGEKKDTCAQECSHFNLTKVESREKLPQPVQVDPVTHCKEKDIDDCWFYFTYSVNGNNEAIVHVVETPDCPTGPDIIPIVAGVVAGIVLIGLALLLIWKLLMIIHDRREFAKFEKEKMNAKWDTGENPIYKSAVTTVVNPKYEGK

SEQ ID NO: 5 - protein, 2477 aa, Homo sapiens
  source 1..2477: note = Wild-type human fibronectin (Uniprot Reference No: P02751-15)
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 6 - protein, 10 aa, synthetic construct
  source 1..10: note = A2-M5-Low, A2-M5-Low+Moderate VH CDR1 (Exemplary, AbM)
GYTFTIYWIN

SEQ ID NO: 7 - protein, 17 aa, synthetic construct
  source 1..17: note = A2-M5-Low VH CDR2 (Exemplary, Kabat)
KIYPGSISTDYNEKFKG

SEQ ID NO: 8 - protein, 17 aa, synthetic construct
  source 1..17: note = A2-M5-Low+Moderate VH CDR2 (Exemplary, Kabat)
KIYPGSISTDYNQKFQG

SEQ ID NO: 9 - protein, 8 aa, synthetic construct
  source 1..8: note = A2-M5-Low, A2-M5-Low+Moderate VH CDR3 (Exemplary, Kabat, AbM)
TGTGGLAY

SEQ ID NO: 10 - protein, 10 aa, synthetic construct
  source 1..10: note = A2-M5-Low, A2-M5-Low+Moderate VL CDR1 (Exemplary, Kabat, AbM
RASSSVNYMY

SEQ ID NO: 11 - protein, 7 aa, synthetic construct
  source 1..7: note = A2-M5-Low, A2-M5-Low+Moderate VL CDR2 (Exemplary, Kabat, AbM)
FTSSRAP

SEQ ID NO: 12 - protein, 9 aa, synthetic construct
  source 1..9: note = A2-M5-Low, A2-M5-Low+Moderate VL CDR3 (Exemplary, IMGT, Kabat, AbM)
QQFTTSPFT

SEQ ID NO: 13 - protein, 8 aa, synthetic construct
  source 1..8: note = A2-M5-Low, A2-M5-Low+Moderate VH CDR1 (IMGT)
GYTFTIYW

SEQ ID NO: 14 - protein, 8 aa, synthetic construct
  source 1..8: note = A2-M5-Low, A2-M5-Low+Moderate VH CDR2 (IMGT)
IYPGSIST

SEQ ID NO: 15 - protein, 10 aa, synthetic construct
  source 1..10: note = A2-M5-Low, A2-M5-Low+Moderate VH CDR3 (IMGT)
AITGTGGLAY

SEQ ID NO: 16 - protein, 5 aa, synthetic construct
  source 1..5: note = A2-M5-Low, A2-M5-Low+Moderate VL CDR1 (IMGT)
SSVNY

SEQ ID NO: 17 - skipped

SEQ ID NO: 18 - protein, 5 aa, synthetic construct
  source 1..5: note = A2-M5-Low, A2-M5-Low+Moderate VH CDR1 (Kabat)
IYWIN

SEQ ID NO: 19 - protein, 7 aa, synthetic construct
  source 1..7: note = A2-M5-Low, A2-M5-Low+Moderate VH CDR1 (Chothia)
GYTFTIY

SEQ ID NO: 20 - protein, 4 aa, synthetic construct
  source 1..4: note = A2-M5-Low, A2-M5-Low+Moderate VH CDR2 (Chothia)
PGSI

SEQ ID NO: 21 - protein, 6 aa, synthetic construct
  source 1..6: note = A2-M5-Low, A2-M5-Low+Moderate VH CDR3 (Chothia)
GTGGLA

SEQ ID NO: 22 - protein, 6 aa, synthetic construct
  source 1..6: note = A2-M5-Low, A2-M5-Low+Moderate VL CDR1 (Chothia)
SSSVNY

SEQ ID NO: 23 - protein, 6 aa, synthetic construct
  source 1..6: note = A2-M5-Low, A2-M5-Low+Moderate VL CDR3 (Chothia)
FTTSPF

SEQ ID NO: 24 - protein, 6 aa, synthetic construct
  source 1..6: note = A2-M5-Low, A2-M5-Low+Moderate VH CDR1 (Contact)
TIYWIN

SEQ ID NO: 25 - protein, 13 aa, synthetic construct
  source 1..13: note = A2-M5-Low, A2-M5-Low+Moderate VH CDR2 (Contact)
WIGKIYPGSISTD

SEQ ID NO: 26 - protein, 9 aa, synthetic construct
  source 1..9: note = A2-M5-Low, A2-M5-Low+Moderate VH CDR3 (Contact)
AITGTGGLA

SEQ ID NO: 27 - protein, 6 aa, synthetic construct
  source 1..6: note = A2-M5-Low, A2-M5-Low+Moderate VL CDR1 (Contact)
NYMYWY

SEQ ID NO: 28 - protein, 10 aa, synthetic construct
  source 1..10: note = A2-M5-Low, A2-M5-Low+Moderate VL CDR2 (Contact)
LWIYFTSSRA

SEQ ID NO: 29 - protein, 8 aa, synthetic construct
  source 1..8: note = A2-M5-Low, A2-M5-Low+Moderate VL CDR3 (Contact)
QQFTTSPF

SEQ ID NO: 30 - protein, 10 aa, synthetic construct
  source 1..10: note = A2-M5-Low, A2-M5-Low+Moderate VH CDR2 (AbM)
KIYPGSISTD

SEQ ID NO: 31 - protein, 117 aa, synthetic construct
  source 1..117: note = A2-M5-Low VH Sequence
QVQLVQPGAEVVKPGASVKLSCKASGYTFTIYWINWVKQAPGQGLEWIGKIYPGSISTDYNEKFKGKATLTVDTSTSTAYMELSSLRSEDTAVYYCAITGTGGLAYWGQGTLVTVSS

SEQ ID NO: 32 - protein, 117 aa, synthetic construct
  source 1..117: note = A2-M5-Low+Moderate VH Sequence
QVQLVQPGAEVKKPGASVKLSCKASGYTFTIYWINWVRQAPGQGLEWIGKIYPGSISTDYNQKFQGRATLTVDTSTSTAYMELSSLRSEDTAVYYCAITGTGGLAYWGQGTLVTVSS

SEQ ID NO: 33 - protein, 106 aa, synthetic construct
  source 1..106: note = A2-M5-Low VL Sequence
ENVLTQSPGIMSASLGERVTMSCRASSSVNYMYWYQQKSGQSPKLWIYFTSSRAPGVPDRFSGSGSGTDYTLTISRMEGEDFATYYCQQFTTSPFTFGQGTKLEIK

SEQ ID NO: 34 - protein, 106 aa, synthetic construct
  source 1..106: note = A2-M5-Low+Moderate VL Sequence
ENVLTQSPGILSASLGERVTMSCRASSSVNYMYWYQQKSGQAPRLWIYFTSSRAPGVPDRFSGSGSGTDYTLTISRMEGEDFATYYCQQFTTSPFTFGQGTKLEIK

SEQ ID NO: 35 - protein, 222 aa, Homo sapiens
  source 1..222: note = Exemplary native human IgG1 Fc region
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 330 aa, Homo sapiens [sequence omitted]

SEQ ID NO: 37 - protein, 222 aa, Homo sapiens
  source 1..222: note = Exemplary native human IgG4 Fc region
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 222 aa, Homo sapiens
  source 1..222: note = Exemplary variant human IgG1 Fc region
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 330 aa, Homo sapiens
  source 1..330: note = Exemplary human IgG1 heavy chain constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 222 aa, Homo sapiens
  source 1..222: note = Exemplary variant human IgG1 Fc region
CPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 222 aa, Homo sapiens
  source 1..222: note = Exemplary variant human IgG1 Fc region
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 222 aa, Homo sapiens
  source 1..222: note = Exemplary variant human IgG1 Fc region
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 222 aa, Homo sapiens
  source 1..222: note = Exemplary variant human IgG1 Fc region
CPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 222 aa, Homo sapiens
  source 1..222: note = Exemplary variant human IgG4 Fc region
CPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 327 aa, Homo sapiens
  source 1..327: note = Exemplary human IgG4 heavy chain constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 107 aa, synthetic construct
  source 1..107: note = Exemplary light chain constant domain
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0193361 A1
(43) Pub. Date: Jul. 9, 2026

(54) ANTI-ALPHA5 INTEGRIN ANTIBODIES AND USES THEREOF

(71) Applicant: PASITHEA THERAPEUTICS CORP., Miami, FL (US)

(72) Inventors: Lisa RYNER, Pacifica, CA (US); Michael LEVITEN, Redwood City, CA (US)

(73) Assignee: PASITHEA THERAPEUTICS CORP., Miami, FL (US)

(21) Appl. No.: 19/128,097

(22) PCT Filed: Nov. 10, 2023
(86) PCT No.: PCT/US2023/079345

§ 371 Date: May 7, 2025

Related U.S. Application Data
(60) Provisional application No. 63/424,820, filed on Nov. 11, 2022.

(51) Int. Cl. C07K 16/28; A61K 47/68; G01N 33/577; G01N 33/68

(52) U.S. Cl. CPC C07K 16/2842; A61K 47/6849; G01N 33/577; G01N 33/6854; C07K 2317/24; C07K 2317/31; C07K 2317/56; C07K 2317/76; G01N 2333/7055

(57) ABSTRACT

The present disclosure provides antibodies or antigen-binding fragments thereof that bind to α5 integrin (e.g., human α5 integrin) and uses thereof. In certain embodiments, the disclosed antibodies are humanized antibodies.

### CROSS-REFERENCE TO RELATED APPLICATIONS

This application claims the benefit of U.S. Provisional Patent Application No. 63/424,820, filed Nov. 11, 2022, the disclosure of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14763-004-228_SEQ LISTING.xml”, was created on Nov. 7, 2023, and is 58,582 bytes in size.

### 1. TECHNICAL FIELD

The present disclosure generally relates to antibodies or antigen-binding fragments thereof that bind to α5 integrin (e.g., human α5 integrin) and uses thereof. In certain embodiments, the disclosed antibodies are humanized antibodies.

### 2. BACKGROUND

Integrins are transmembrane proteins that bind extracellular matrix (ECM) components and regulate cell adhesion, migration and activation. Each integrin is composed of an α and a β transmembrane integrin subunit. There are 18 α integrin subunits and 8 β integrin subunits in the human genome and they combine to generate 24 unique heterodimeric integrins. These heterodimers modulate cell behavior through mechanisms known as “inside-out” and “outside-in” signaling. In the former, intracellular proteins bind the integrin cytoplasmic domain and stabilizes a conformation that binds extracellular ligands with high affinity. Then, through “outside-in” signaling the ligand-bound integrin stimulates intracellular signaling cascades that modulate cell behaviors.

The α5β1 integrin is known as the fibronectin (FN) receptor because of its high affinity for the FN in the extracellular matrix (ECM). This binding is mediated by the ligand-binding site at the interface between the α and β subunits in the headpiece of α5β1 and an arginine-glycine-aspartic acid (RGD) peptide motif in the Type III repeats of FN. The α5β1 integrin binds additional RGD-containing proteins like osteopontin and fibrillin along with proteins that lack RGD motifs including CD40L, IL-1b and the TNF-α converting enzyme ADAM-17. Consistent with the tissue distribution of its ligands, α5β1 is expressed by a variety of cell-types including endothelial cells, mast cells and macrophage lineages in peripheral tissues and the central nervous system (CNS) (e.g., microglia and perivascular macrophages).

The association of α5β1 integrin with tumor angiogenesis is well-established. In addition, α5β1 has been demonstrated to be present on tumor cells. Antibodies that bind α5β1 have been shown not only to inhibit angiogenesis but also facilitate killing of α5β1 expressing tumor cells. The association of α5β1 integrin with neuroinflammatory diseases including multiple sclerosis (MS) and amyotrophic lateral sclerosis (ALS) has also been demonstrated. Antibodies that bind to α5β1 have been shown to ameliorate symptoms in the experimental autoimmune encephalitis (EAE) model of MS and the SOD1^{G93A} transgenic model of ALS. Although expression of α5β1 would appear to give it the potential to be a target in anti-angiogenesis and cancer therapies as well as in neuroinflammatory disease therapies, clinical success with antibodies targeting α5 integrin has not yet been achieved.

Accordingly, there remains a need in the art for agents that can target α5β1 integrin to treat, prevent, or alleviate α5-mediated diseases, disorders, or conditions, including those involving cells expressing α5β1, such as tumor cells and macrophages.

### 3. SUMMARY OF THE INVENTION

The present disclosure provides antibodies or antigen-binding fragments thereof that bind to α5 integrin (e.g., human α5 integrin). In certain embodiments, the anti-α5 integrin antibody or an antigen-binding fragment thereof comprises a heavy chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31. In certain embodiments, the heavy chain variable region comprises the amino acid sequence set forth in SEQ ID NO: 31.

In certain embodiments, the anti-α5 integrin antibody or antigen-binding fragment thereof comprises a heavy chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 32. In certain embodiments, the heavy chain variable region comprises the amino acid sequence set forth in SEQ ID NO: 32.

In certain embodiments, the anti-α5 integrin antibody or antigen-binding fragment thereof comprises a light chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33. In certain embodiments, the light chain variable region comprises the amino acid sequence set forth in SEQ ID NO: 33.

In certain embodiments, the anti-α5 integrin antibody or antigen-binding fragment thereof comprises a light chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 34. In certain embodiments, the light chain variable region comprises the amino acid sequence set forth in SEQ ID NO: 34.

In certain embodiments, the anti-α5 integrin antibody or antigen-binding fragment thereof comprising: (a) a heavy chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31 or SEQ ID NO: 32; and (b) a light chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33 or SEQ ID NO: 34. In certain embodiments, (a) the heavy chain variable region comprises the amino acid sequence set forth in SEQ ID NO: 31 or SEQ ID NO: 32; and (b) the light chain variable region comprises the amino acid sequence set forth in SEQ ID NO: 33 or SEQ ID NO: 34.

In certain embodiments, (a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31; and (b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33. In certain embodiments, (a) the heavy chain variable region comprises the amino acid sequence set forth in SEQ ID NO: 31; and (b) the light chain variable region comprises the amino acid sequence set forth in SEQ ID NO: 33.

In certain embodiments, (a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31; and (b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 34. In certain embodiments, (a) the heavy chain variable region comprises the amino acid sequence set forth in SEQ ID NO: 31; and (b) the light chain variable region comprises the amino acid sequence set forth in SEQ ID NO: 34.

In certain embodiments, (a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 32; and (b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33. In certain embodiments, (a) the heavy chain variable region comprises the amino acid sequence set forth in SEQ ID NO: 32; and (b) the light chain variable region comprises the amino acid sequence set forth in SEQ ID NO: 33.

In certain embodiments, (a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 32; and (b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 34. In certain embodiments, (a) the heavy chain variable region comprises the amino acid sequence set forth in SEQ ID NO: 32; and (b) the light chain variable region comprises the amino acid sequence set forth in SEQ ID NO: 34.

In certain embodiments, the antibody is a monoclonal antibody. In certain embodiments, the antibody is a humanized antibody. In certain embodiments, the antigen-binding fragment is a Fab, Fab′, F(ab′)₂, variable fragment (Fv), or single chain variable fragment (scFv).

In certain embodiments, the antibody or antigen-binding fragment thereof binds to α5β1 integrin. In certain embodiments, the antibody or antigen-binding fragment thereof inhibits the binding of α5β1 integrin to fibronectin.

The present disclosure further provides antibodies or antigen-binding fragments thereof that compete with the any of the disclosed antibodies or antigen-binding fragments thereof for binding to α5 integrin.

The present disclosure further provides antibodies or antigen-binding fragments thereof that binds to essentially the same epitope region on as any of the disclosed antibodies or antigen-binding fragments thereof for binding to α5 integrin.

Further provided are compositions comprising the disclosed antibodies or antigen-binding fragments thereof. In certain embodiments, the composition is a pharmaceutical composition comprising a pharmaceutically acceptable carrier.

The present disclosure further provides conjugates comprising the disclosed antibodies or antigen-binding fragments thereof. In certain embodiments, the antibody or antigen-binding fragment thereof is linked to a therapeutic agent, a detectable agent, or a diagnostic agent. In certain embodiments, the conjugate is an immunoconjugate. In certain embodiments, the therapeutic agent is a chemotherapeutic agent, a cytotoxin, or a drug. Also provided are compositions comprising the disclosed conjugates. In certain embodiments, the composition is a pharmaceutical composition comprising a pharmaceutically acceptable carrier.

The present disclosure further provides multispecific molecules comprising the disclosed antibodies or antigen-binding fragments thereof, linked to a second functional moiety. In certain embodiments, the second functional moiety has a different binding specificity than the antibody or antigen binding fragment thereof. Also provided are compositions comprising the disclosed multispecific molecules. In certain embodiments, the composition is a pharmaceutical composition comprising a pharmaceutically acceptable carrier.

Further provided are nucleic acids encoding the disclosed antibodies or antigen-binding fragments thereof. In certain embodiments, the nucleic acid comprises a first polynucleotide that encodes a V_{H} comprising the amino acid sequence set forth in SEQ ID NO: 31 or SEQ ID NO: 32; and/or a second polynucleotide that encodes a V_{L} comprising the amino acid sequence set forth in SEQ ID NO: 33 or SEQ ID NO: 34. In certain embodiments, the first polynucleotide and the second polynucleotide are selected from the group consisting of:
(a) a first polynucleotide that encodes a V_{H} comprising the amino acid sequence set forth in SEQ ID NO: 31; and a second polynucleotide that encodes a V_{L} comprising the amino acid sequence set forth in SEQ ID NO: 33;
(b) a first polynucleotide that encodes a V_{H} comprising the amino acid sequence set forth in SEQ ID NO: 31; and a second polynucleotide that encodes a V_{L} comprising the amino acid sequence set forth in SEQ ID NO: 34;
(c) a first polynucleotide that encodes a V_{H} comprising the amino acid sequence set forth in SEQ ID NO: 32; and a second polynucleotide that encodes a V_{L} comprising the amino acid sequence set forth in SEQ ID NO: 33; and
(d) a first polynucleotide that encodes a V_{H} comprising the amino acid sequence set forth in SEQ ID NO: 32; and a second polynucleotide that encodes a V_{L} comprising the amino acid sequence set forth in SEQ ID NO: 34.

Further provided are vectors comprising the disclosed nucleic acid. In certain embodiments, the vector is an expression vector. Further provided are host cells comprising the disclosed vectors.

The present discourse also provides methods of producing the disclosed anti-α5 integrin antibodies or antigen-binding fragments thereof. In certain embodiments, the method comprises culturing a presently disclosed host cell under conditions to induce expression of the antibody or antigen-binding fragment thereof from the host cell.

The present discourse further provides methods for detecting α5 integrin in a whole cell or tissue. In certain embodiments, the method comprises contacting a cell or tissue with the disclosed antibody or antigen-binding fragment thereof, wherein the antibody or antigen-binding fragment thereof comprises a detectable label; and determining the amount of the labeled antibody or antigen-binding fragment thereof bound to the cell or tissue by measuring the amount of detectable label associated with said cell or tissue, wherein the amount of bound antibody or antigen-binding fragment thereof indicates the amount of α5 integrin in said cell or tissue.

The present discourse further provides methods for treating an α5 integrin-associated disease, disorder or condition in a subject. In certain embodiments, the method comprises administering to the subject the presently disclosed antibody or antigen-binding fragment thereof, multispecific molecule, conjugate, or composition. The present discourse further provides the presently disclosed antibody or antigen-binding fragment thereof, multispecific molecule, conjugate, or composition for use in treating an α5 integrin-associated disease, disorder or condition in a subject.

In certain embodiments, the α5 integrin-associated disease, disorder or condition is associated with α5β1. In certain embodiments, the α5 integrin-associated disease, disorder or condition is a tumor. In certain embodiments, the tumor is a solid tumor. In certain embodiments, the tumor is cancer. In certain embodiments, the cancer is selected from the group consisting of breast cancer, bladder cancer, melanoma, prostate cancer, mesothelioma, lung cancer, brain cancer, ovarian cancer, colon cancer, testicular cancer, thyroid cancer, squamous cell carcinoma, glioblastoma, neuroblastoma, neurofibromatosis, uterine cancer, colorectal cancer, stomach cancer, and pancreatic cancer.

In certain embodiments, the α5 integrin-associated disease, disorder or condition is associated with abnormal angiogenesis. In certain embodiments, the α5 integrin-associated disease, disorder or condition is an ocular disease. In certain embodiments, the ocular disease is selected from the group consisting of diabetic retinopathy, age-induced macular degeneration, and uveitis.

In certain embodiments, the α5 integrin-associated disease, disorder or condition is an inflammatory disease, disorder or condition. In certain embodiments, the inflammatory disease, disorder, or condition is a neuroinflammatory disease, disorder or condition. In certain embodiments, the inflammatory disease, disorder or condition is selected from the group consisting of macrophages-mediated innate immunity diseases, multiple sclerosis, amyotrophic lateral sclerosis (ALS), and atherosclerosis.

The present discourse further provides methods for inhibiting abnormal angiogenesis in a subject. In certain embodiments, the method comprising administering to the subject the presently disclosed antibody or antigen-binding fragment thereof, multispecific molecule, conjugate, or composition. The present discourse further provides the presently disclosed antibody or antigen-binding fragment thereof, multispecific molecule, conjugate, or composition for use in inhibiting abnormal angiogenesis in a subject. In certain embodiments, the subject suffers from a tumor.

For various methods disclosed herein, in certain embodiments, the subject is human.

### 4. DETAILED DESCRIPTION

The present disclosure provides antibodies or antigen-binding fragments thereof that bind to α5 integrin (e.g., human α5 integrin). In certain embodiments, the disclosed antibodies are humanized antibodies. In certain embodiments, the anti-α5 integrin antibodies or antigen-binding fragments thereof disclosed herein are capable of inhibiting the binding of α5β1 integrin to fibronectin.

The present disclosure further provides compositions comprising the anti-α5 integrin antibodies or antigen-binding fragments thereof disclosed herein. In certain embodiments, the compositions are pharmaceutical compositions comprising a pharmaceutically acceptable carrier. Also provided are multispecific molecules comprising the anti-α5 integrin antibodies or antigen-binding fragments thereof disclosed herein. In addition, the present disclosure provides conjugates comprising the anti-α5 integrin antibodies or antigen-binding fragments thereof disclosed herein.

The present disclosure also provides methods of treating a disease, disorder, or condition associated with α5 integrin using the presently disclosed anti-α5 integrin antibodies or antigen-binding fragments thereof, multispecific molecules, compositions, or conjugates. In certain embodiments, the disease, disorder, or condition is associated with α5β1 integrin (referred to as “an α5β1 integrin-associated disease, disorder, and condition”. Non-limiting examples of α5β1 integrin-associated diseases, disorders, and conditions include cancer, angiogenesis-associated diseases (e.g., a disease with abnormal angiogenesis), and an inflammatory disease (e.g., a neuroinflammatory disease).

Non-limiting embodiments of the present disclosure are described by the present specification and Examples.

For purposes of clarity of disclosure and not by way of limitation, the detailed description is divided into the following subsections:
4.1. Definitions;
4.2. Integrins;
4.3. Anti-α5 integrin Antibodies and Antigen-binding Fragments thereof;
4.4 Nucleic Acids Encoding the Antibodies and Vectors;
4.5. Methods of Detection;
4.6 Compositions and Formulations;
4.7. Methods of Treatment; and
4.8 Kits.

### 4.1. Definitions

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full length monoclonal antibodies), antibody compositions with polyepitopic or monoepitopic specificity, recombinantly produced antibodies, monospecific antibodies, multispecific antibodies (including bispecific antibodies), synthetic antibodies, chimeric antibodies, humanized antibodies, or human versions of antibodies having full length heavy and/or light chains. The present disclosure also includes antibody fragments (and/or polypeptides that comprise antibody fragments) that retain α5 integrin binding characteristics. Non-limiting examples of antibody fragments include antigen-binding regions and/or effector regions of the antibody, e.g., Fab, Fab′, F(ab′)2, Fv, scFv, (scFv)₂, single chain antibody molecule, dual variable region antibody, single variable region antibody, linear antibody, V region, a multispecific antibody formed from antibody fragments, F(ab)₂, Fd, Fc, diabody, di-diabody, disulfide-linked Fvs (dsFv), single-domain antibody (e.g., VHH, nanobody) or other fragments (e.g., fragments consisting of the variable regions of the heavy and light chains that are non-covalently coupled). In general terms, a variable region domain may be any suitable arrangement of immunoglobulin heavy (V_{H}) and/or light (V_{L}) variable regions. For example, the present disclosure also includes tetrameric antibodies comprising two heavy chain and two light chain molecules, an antibody light chain monomer, and an antibody heavy chain monomer. Thus, for example, the variable region domain may be dimeric and contain V_{H}—V_{H}, V_{H}-V_{L}, or V_{L}-V_{L} dimers that bind α5 integrin. If desired, the V_{H} and V_{L} chains may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). The antibodies or antigen-binding fragments thereof, in certain embodiments, comprise a light chain and/or a heavy chain constant region, such as one or more constant regions, including one or more IgG1, IgG2, IgG3 and/or IgG4 constant regions. In certain embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies can be antagonistic antibodies or agonistic antibodies.

As used herein, the term “monospecific antibody” refers to an antibody that has one or more binding sites each of which bind to the same epitope of the same antigen, e.g., α5 integrin (e.g., human α5 integrin).

As used herein, the term “multispecific molecule” refers to a molecule (e.g., an antibody) that is capable of binding to at least two distinct antigenic determinants, for example two binding sites each formed by a pair of an antibody heavy chain variable domain (V_{H}) and an antibody light chain variable domain (V_{L}) binding to different antigens or to different epitopes on the same antigen. Such a multispecific molecule may have a 1+1 format. Other multispecific molecule formats may be 2+1 or 1+2 formats (comprising two binding sites for a first antigen or epitope and one binding site for a second antigen or epitope) or 2+2 formats (comprising two binding sites for a first antigen or epitope and two binding sites for a second antigen or epitope). When a multispecific molecule comprises two antigen binding sites, each may bind to a different antigenic determinant. Such a multispecific molecule may bind to two different epitopes on the same antigen (e.g., epitopes on α5 integrin) or on different antigens (e.g., an epitope on α5 integrin and an epitope on a non-α5 integrin).

The terms “identical” or percent “identity” in the context of two or more nucleic acids or polynucleotides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In certain embodiments, two nucleic acids or polypeptides are substantially identical, meaning they have at least about 70%, at least about 75%, at least about 80%, at least about 85%, at least about 90%, and in certain embodiments at least about 95%, at least about 96%, at least about 97%, at least about 98%, at least about 99% nucleotide or amino acid residue identity, when compared and aligned for maximum correspondence, as measured using a sequence comparison algorithm or by visual inspection. In certain embodiments, identity exists over a region of the amino acid sequences that is at least about 10 residues, at least about 20 residues, at least about 40-60 residues, at least about 60-80 residues in length or any integral value there between. In certain embodiments, identity exists over a longer region than 60-80 residues, such as at least about 80-100 residues, and in certain embodiments the sequences are substantially identical over the full length of the sequences being compared, such as the coding region of a target protein or an antibody. In certain embodiments, identity exists over a region of the nucleotide sequences that is at least about 10 bases, at least about 20 bases, at least about 40-60 bases, at least about 60-80 bases in length or any integral value there between. In certain embodiments, identity exists over a longer region than 60-80 bases, such as at least about 80-1000 bases or more, and in certain embodiments the sequences are substantially identical over the full length of the sequences being compared, such as a nucleotide sequence encoding a protein of interest.

As used herein, the term “conservative modifications” refers to an amino acid modification that does not significantly affect or alter the binding characteristics of the antibody containing the amino acid sequence. Such conservative modifications include amino acid substitutions, additions and deletions. Modifications can be introduced into a presently disclosed antibody or antigen-binding fragment thereof by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid substitutions may be introduced into an antibody of interest and the products screened for a desired activity, e.g., retained/improved antigen binding, decreased immunogenicity, or improved ADCC or CDC. For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art. In certain embodiments, an amino acid sequence disclosed herein, e.g., a CDR sequence, a heavy chain variable region (V_{H}) sequence or a light chain variable region (V_{L}) sequence, can have up to about one, up to about two, up to about three, up to about four, up to about five, up to about six, up to about seven, up to about eight, up to about nine or up to about ten amino acid residues that are modified and/or substituted.

The terms “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component. Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in certain embodiments, the polypeptides can occur as single chains.

As used herein, an “epitope” is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human α5β1 integrin. A linear epitope may or may not be dependent on secondary, tertiary, or quaternary structure. For example, in certain embodiments, an antibody binds to a group of amino acids regardless of whether they are folded in a natural three dimensional protein structure. In certain embodiments, an antibody requires amino acid residues making up the epitope to exhibit a particular conformation (e.g., bend, twist, turn or fold) in order to recognize and bind the epitope.

An antibody or an antigen-binding fragment thereof binds to “an epitope region”, “an epitope” or “essentially the same epitope” or “the same epitope” as a reference antibody or a reference antigen-binding fragment thereof, when the two recognize identical, overlapping or adjacent epitopes or epitope regions in a three-dimensional space. The most widely used and rapid methods for determining whether two antibodies or two antigen-binding fragments bind to identical, overlapping or adjacent epitopes or epitope regions in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, for example, using competition assays. Such assays can be combined with computational processes for clustering antibodies based on their epitope recognition properties and identifying antibodies having distinct binding specificities.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In certain embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, ID), or other assays known in the art. In certain embodiments, an antibody or an antigen-binding fragment thereof binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. In certain embodiments, molecules that specifically bind to an antigen bind to the antigen with a Ka that is at least about 2 logs, at least about 2.5 logs, at least about 3 logs, at least about 4 logs or greater than the Ka when the molecules bind to another antigen. In certain embodiments, molecules that specifically bind to an antigen do not cross react with other proteins. In certain embodiments, molecules that specifically bind to an antigen do not cross react with other non-α5β1 integrin proteins. In certain embodiments “specifically binds” means, for instance, that a polypeptide or molecule binds a protein or target with a K_{D} of about 0.1 mM or less, but more usually less than about 1 μM. In certain embodiments, “specifically binds” means that a polypeptide or molecule binds a target with a K_{D} of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in certain embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in certain embodiments, specifically bind to more than one target. In certain embodiments, multiple targets can be bound by the same antigen-binding site on the polypeptide or molecule. For example, an antibody can, in certain instances, comprise two identical antigen-binding sites, each of which specifically binds the same epitope on two or more proteins. In some alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In certain embodiments, the “K_{D}” or “K_{D} value” may be measured by biolayer interferometry (BLI) using, for example, the OctetQK384 system (ForteBio, Menlo Park, CA). Alternatively or additionally, the K_{D} may also be measured in a radiolabeled antigen binding assay (RIA), for example, performed with the Fab version of an antibody of interest and its antigen (Chen, et al., (1999) J. Mol Biol 293:865-881) or using surface plasmon resonance (SPR) assays by Biacore, using, for example, a BIAcore™-2000 or a BIAcore™-3000 BIAcore, Inc., Piscataway, NJ). An “on-rate” or “rate of association” or “association rate” or “kₒₙ,” as well as an “off-rate” or “rate of dissociation” or “dissociation rate” or “k_{off},” may also be determined with the same SPR or BLI techniques described above using, for example, the OctetQK384 sytem (ForteBio, Menlo Park, CA) or a BIAcore™-2000 or a BIAcore™-3000 (BIAcore, Inc., Piscataway, NJ), respectively.

As used herein, the term “constant region” or “constant domain” is a well-known antibody term of art and refers to an antibody portion, e.g., for example, a carboxyl terminal portion of a light and/or heavy chain which is not directly involved in binding of an antibody to an antigen but which can exhibit various effector functions, such as interaction with the Fc receptor. The term includes the portion of an immunoglobulin molecule having a generally more conserved amino acid sequence relative to an immunoglobulin variable domain.

Antibody “effector functions” refer to those biological activities attributable to the Fc region (e.g., a native sequence Fc region or amino acid sequence variant Fc region) of an antibody, and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) of lambda (λ) based on the amino acid sequence of the constant domains. Light chain amino acid sequences are well known in the art.

The terms “antigen-binding fragment” refers to that portion of an antibody, which comprises the amino acid residues that interact with an antigen and confer on the binding fragment, domain, or region its specificity and affinity for the antigen (e.g., the CDRs). “Antigen binding fragment” as used herein includes “antibody fragment,” which comprises a portion of an antibody including one or more CDRs, such as the antigen binding or variable region of the antibody.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above. In certain embodiments, the antibodies are humanized antibodies.

In certain embodiments, the antibodies described herein include immunoglobulin molecules and immunologically active portions of immunoglobulin molecules, including molecules that contain one or more antigen binding sites that bind to an α5 integrin.

Antibodies can be of any type (e.g., IgG, IgE, IgM, IgD, IgA or IgY), any class, (e.g., IgG1, IgG2, IgG3, IgG4, IgAQ1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

In certain embodiments, an antibody is a 4-chain antibody unit comprising two heavy (H) chain/light (L) chain pairs, wherein the amino acid sequences of the H chains are identical and the amino acid sequences of the L chains are identical. In certain embodiments, the H and L chains comprise constant regions, for example, human constant regions. In certain embodiments, the L chain constant region of such antibodies is a kappa or lambda light chain constant region, for example, a human kappa or lambda light chain constant region. In certain embodiments, the H chain constant region of such antibodies comprise a gamma heavy chain constant region, for example, a human gamma heavy chain constant region. In certain embodiments, such antibodies comprise IgG constant regions, for example, human IgG constant regions (e.g., IgG1, IgG2, IgG3, and/or IgG4 constant regions).

The term “variable region” or “variable domain” refers to a portion of the light or heavy chains of an antibody that is generally located at the amino-terminal of the light or heavy chain, and are used in the binding and specificity of each particular antibody for its particular antigen. The variable region of the heavy chain may be referred to as “V_{H}.” The variable region of the light chain may be referred to as “V_{L}.” The term “variable” refers to the fact that certain segments of the variable regions differ extensively in sequence among antibodies. The V region mediates antigen binding and defines specificity of a particular antibody for its particular antigen. However, the variability is not evenly distributed across the variable regions. Instead, the V regions consist of less variable (e.g., relatively invariant) stretches called framework regions (FRs) separated by shorter regions of greater variability (e.g., extreme variability) called “hypervariable regions” or alternatively called “complementarity determining regions” (“CDRs”). The variable regions of heavy and light chains each comprise four FRs (FR1, FR2, FR3 and FR4), largely adopting a β sheet configuration, connected by three hypervariable regions, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5^{th} Ed. Public Health Service, National Institutes of Health, Bethesda, MD, (1991)). The constant regions are not involved directly in binding an antibody to an antigen, but may exhibit various effector functions, such as participation of the antibody in antibody dependent cellular cytotoxicity (ADCC) and complement dependent cytotoxicity (CDC). The CDRs of the light and heavy chains are primarily responsible for the interaction of the antibody with antigen.

Generally, an antibody comprises six CDR regions: three in the V_{H} (HCDR1, HCDR2, and HCDR3), and three in the V_{L} (LCDR1, LCDR2, and LCDR3). CDRs can be identified according to a number of known numbering systems. In certain embodiments, the CDRs are identified according to the Kabat numbering system. The Kabat CDRs are based on sequence variability and are the most commonly used (see e.g., Kabat et al., Sequences of Proteins of Immunological Interest, 5^{th} Ed. Public Health Service, National Institutes of Health, Bethesda, MD. (1991)). In certain embodiments, the CDRs are identified according to the Chothia numbering system. Chothia refers to the location of the structural loops (see e.g., Chothia and Lesk, J. Mol. Biol. 196:901-917 (1987)). In certain embodiments, the CDRs are identified according to the AbM numbering system. The AbM hypervariable regions represent a compromise between the Kabat CDRs and Chothia structural loops, and are used by Oxford Molecular's AbM antibody modeling software (see e.g., Martin, in Antibody Engineering, Vol. 2, Chapter 3, Springer Verlag). In certain embodiments, the CDRs are identified according to the Contact numbering system (see, e.g., MacCallum R M et al., 1996, J Mol Biol 5:732-745). The Contact CDRs are based on an analysis of the available complex crystal structures. In certain embodiments, the CDRs are identified according to the ImMunoGeneTics (IMGT) Information System®. A universal numbering system has been developed and widely adopted, ImMunoGeneTics (IMGT) Information System® (Lefranc et al., Dev. Comp. Immunol. 27 (1): 55-77 (2003)). IMGT is an integrated information system specializing in immunoglobulins (IG), T cell receptors (TR) and major histocompatibility complex (MHC) of human and other vertebrates.

Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (Ahon) has been developed by Honegger and Plückthun, J. Mol. Biol. 309:657-670 (2001). Correspondence between the numbering system, including, for example, the Kabat numbering and the IMGT unique numbering system, is well known to one skilled in the art (see e.g., Kabat, supra; Chothia and Lesk, supra; Martin, supra; Lefranc et al., supra) and is also illustrated below. An Exemplary system, shown herein, identifies CDRs as the longer sequences of Kabat and AbM. An exemplary demonstrations of the various numbering systems are provided in Table 1.

TABLE 1
Antibody CDR numbering systems

| | Exem- | | | | | |
|---|---|---|---|---|---|---|
| | plary | IMGT | Kabat | AbM | Chothia | Contact |
| V_{H} | 26-35 | 27-38 | 31-35 | 26-35 | 26-32 | 30-35 |
| CDR1 | | | | | | |
| V_{H} | 50-65 | 56-65 | 50-65 | 50-58 | 52a/53-55 | 47-58 |
| CDR2 | | | | | | |
| V_{H} | 95-102 | 105-117 | 95-102 | 95-102 | 96-101 | 93-101 |
| CDR3 | | | | | | |
| V_{L} | 24-34 | 27-38 | 24-34 | 24-34 | 26-32 | 30-36 |
| CDR1 | | | | | | |
| V_{L} | 50-56 | 56-65 | 50-56 | 50-56 | 50-52 | 46-55 |
| CDR2 | | | | | | |
| V_{L} | 89-97 | 105-117 | 89-97 | 89-97 | 91-96 | 89-96 |
| CDR3 | | | | | | |

Hypervariable regions may comprise “extended hypervariable regions” as follows: 24-36 or 24-34 (L1), 46-56 or 50-56 (L2) and 89-97 or 89-96 (L3) in the V_{L} and 26-35 or 26-35A (H1), 50-65 or 49-65 (H2) and 93-102, 94-102, or 95-102 (H3) in the V_{H}. As used herein, the terms “hypervariable region,” “HVR,” “HV,” “complementarity determining region,” or “CDR” are used interchangeably.

The term “vector” refers to a substance that is used to carry or include a nucleic acid, including for example, in order to introduce a nucleic acid into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g., both an antibody heavy and light chain or an antibody V_{H} and V_{L}) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acids into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acids are expressed in a sufficient amount to produce a desired product (e.g., an anti-α5 integrin antibody or antigen-binding fragment thereof as described herein), and it is further understood that expression levels can be optimized to obtain sufficient expression using methods well known in the art.

An “effective amount” is generally an amount sufficient to reduce the severity and/or frequency of one or more symptoms, eliminate the one or more symptoms and/or underlying cause, prevent the occurrence of one or more symptoms and/or their underlying cause, and/or improve or remediate the damage that results from or is associated with a disease, disorder, or condition. In certain embodiments, the effective amount is a therapeutically effective amount or a prophylactically effective amount.

The term “therapeutically effective amount” as used herein refers to the amount of an agent (e.g., an antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the prophylactic or therapeutic effect of another therapy (e.g., a therapy other than the administration of an anti-α5 integrin antibody described herein). A “therapeutically effective amount” of a substance/molecule/agent of the present disclosure (e.g., an anti-α5 integrin antibody) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an antibody or other agent (e.g., or drug) effective to “treat” a disease, disorder, or condition, in a subject or mammal.

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s). The full therapeutic or prophylactic effect does not necessarily occur by administration of one dose, and may occur only after administration of a series of doses. Thus, a therapeutically or prophylactically effective amount may be administered in one or more administrations.

“Carriers” as used herein include carriers, excipients, or stabilizers that are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington's Pharmaceutical Sciences (1990) Mack Publishing Co., Easton, PA. Compositions, including pharmaceutical compounds, may contain a prophylactically or therapeutically effective amount of an anti-α5 integrin antibody or antigen-binding fragment thereof, for example, in isolated or purified form, together with a suitable amount of carrier so as to provide the form for proper administration to the subject (e.g., patient). The formulation should suit the mode of administration.

As used herein, an “individual” or a “subject” refers to a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses, and non-human primates such as apes and monkeys. In certain embodiments, the subject is a mammal. In certain embodiments, the subject is a human.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 15%, up to 10%, up to 9%, up to 8%, up to 7%, up to 6%, up to 5%, up to 4%, up to 3%, up to 2%, or up to 1% of a given value or range. Alternatively, particularly with respect to biological systems or processes, the term can mean within an order of magnitude, e.g., within 5-fold or within 2-fold, of a value.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 4.2. Integrins

Integrins are heterodimeric, ubiquitous, and transmembrane glycoprotein receptors, which primarily act as signaling proteins in mammals. Each integrin consists of an α-subunit and a β-subunit. There are 18 variants of α-subunit, and 8 variants of β-subunit, which creates 24 unique heterodimeric integrins. The α- and β-subunits are bound in a noncovalent complex with the ligand-binding site at the interface. These heterodimers modulate cell behavior through mechanisms known as “inside-out” and “outside-in” signaling. In the former, intracellular proteins bind the integrin cytoplasmic domain and stabilizes a conformation that binds extracellular ligands with high affinity. Then, through “outside-in” signaling the ligand-bound integrin stimulates intracellular signaling cascades that modulate cell behaviors.

α5 is also known as A5, Integrin alpha-5, ITGA5 protein, CD49e antigen, Glycoprotein Ic (GPIc), VLA5A, FNRA, and fibronectin receptor subunit alpha. The term “α5 integrin” refers a polypeptide or a protein of any native α5 integrin from any vertebrate source, including mammals such as primates (e.g., humans, cynomolgus monkey (cyno)), dogs, and rodents (e.g., mice and rats). The term “α5 integrin” encompasses “full-length,” unprocessed α5 integrin, as well as any form of α5 integrin or any fragment thereof that results from processing in the cell. The term “α5 integrin” also encompasses naturally occurring variants of α5 integrin, such as SNP variants, splice variants and allelic variants. An α5 integrin in association with a β1 integrin as a heterodimer is known in the art to interact with a number of ligands (e.g., fibronectin) and this interaction leads to protein conformational changes and signal transduction, leading to changes in cellular activity, such as cell adhesion, proliferation, apoptosis, migration, and phagocytosis. The α5β1 integrin is known as the fibronectin receptor due to its high affinity for the fibronectin in the extracellular matrix (ECM). This binding is mediated by the ligand-binding site at the interface between the α and β subunits in the headpiece of α5β1 and an arginine-glycine-aspartic acid (RGD) peptide motif in the Type III repeats of fibronectin. In addition to fibronectin, α5β1 integrin binds to additional RGD-containing proteins, e.g., osteopontin and fibrillin along with proteins that lack RGD motifs, e.g., CD40L, IL-1b and the TNF-α converting enzyme ADAM-17. Consistent with the tissue distribution of its ligands, α5β1 is expressed by a variety of cell-types including endothelial cells, mast cells and macrophage lineages in peripheral tissues and the central nervous system (CNS) (e.g., microglia and perivascular macrophages).

In certain embodiments, the presently disclosed anti-α5 integrin antibody or antigen-binding fragment thereof binds to a human α5 integrin. In certain embodiments, the human α5 integrin is a wild-type human α5 integrin or a fragment thereof. In certain embodiments, the wild-type human α5 integrin comprises the amino acid sequence with a Uniprot Reference No: P08648 (SEQ ID NO: 1). SEQ ID NO: 1 is provided below.

(SEQ ID NO: 1)
MGSRTPESPLHAVQLRWGPRRRPPLLPLLLLLLPPPPRVGGFNLDAEAP
AVLSGPPGSFFGFSVEFYRPGTDGVSVLVGAPKANTSQPGVLQGGAVYL
CPWGASPTQCTPIEFDSKGSRLLESSLSSSEGEEPVEYKSLQWFGATVR
AHGSSILACAPLYSWRTEKEPLSDPVGTCYLSTDNFTRILEYAPCRSDF
SWAAGQGYCQGGFSAEFTKTGRVVLGGPGSYFWQGQILSATQEQIAESY
YPEYLINLVQGQLQTRQASSIYDDSYLGYSVAVGEFSGDDTEDFVAGVP
KGNLTYGYVTILNGSDIRSLYNFSGEQMASYFGYAVAATDVNGDGLDDL
LVGAPLLMDRTPDGRPQEVGRVYVYLQHPAGIEPTPTLTLTGHDEFGRF
GSSLTPLGDLDQDGYNDVAIGAPFGGETQQGVVFVFPGGPGGLGSKPSQ
VLQPLWAASHTPDFFGSALRGGRDLDGNGYPDLIVGSFGVDKAVVYRGR
PIVSASASLTIFPAMFNPEERSCSLEGNPVACINLSFCLNASGKHVADS
IGFTVELQLDWQKQKGGVRRALFLASRQATLTQTLLIQNGAREDCREMK
IYLRNESEFRDKLSPIHIALNFSLDPQAPVDSHGLRPALHYQSKSRIED
KAQILLDCGEDNICVPDLQLEVFGEQNHVYLGDKNALNLTFHAQNVGEG
GAYEAELRVTAPPEAEYSGLVRHPGNFSSLSCDYFAVNQSRLLVCDLGN
PMKAGASLWGGLRFTVPHLRDTKKTIQFDFQILSKNLNNSQSDVVSFRL
SVEAQAQVTLNGVSKPEAVLFPVSDWHPRDQPQKEEDLGPAVHHVYELI
NQGPSSISQGVLELSCPQALEGQQLLYVTRVTGLNCTTNHPINPKGLEL
DPEGSLHHQQKREAPSRSSASSGPQILKCPEAECFRLRCELGPLHQQES
QSLQLHFRVWAKTFLQREHQPFSLQCEAVYKALKMPYRILPRQLPQKER
QVATAVQWTKAEGSYGVPLWIIILAILFGLLLLGLLIYILYKLGFFKRS
LPYGTAMEKAQLKPPATSDA

In certain embodiments, the human α5 integrin comprises an extracellular domain, a transmembrane domain, and a cytoplasmic domain. In certain embodiments, the extracellular domain of the human α5 integrin comprises amino acids 42 to 995 of SEQ ID NO: 1. In certain embodiments, the transmembrane domain of the human α5 integrin comprises amino acids 996 to 1021 of SEQ ID NO: 1. In certain embodiments, the cytoplasmic domain of the human α5 integrin comprises amino acids 1022 to 1049 of SEQ ID NO: 1. In certain embodiments, the human α5 integrin comprises a signal peptide. In certain embodiments, the signal peptide of the human α5 integrin comprises amino acids 1 to 41 of SEQ ID NO: 1.

In certain embodiments, the α5 integrin comprises or consists of an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 1 or a fragment thereof, and/or may comprise up to one or up to two or up to three conservative amino acid substitutions.

For example, in certain embodiments, the presently disclosed anti-α5 integrin antibody or antigen-binding fragment thereof binds to the extracellular domain of an α5 integrin (e.g., a human α5 integrin). In certain embodiments, the anti-α5 integrin antibody or antigen-binding fragment thereof binds to a fragment of SEQ ID NO: 1. In certain embodiments, the presently disclosed anti-α5 integrin antibody or antigen-binding fragment thereof binds to amino acids 42 to 995 of SEQ ID NO: 1.

In certain embodiments, the presently disclosed anti-α5 integrin antibody or antigen-binding fragment thereof does bind to a mouse α5 integrin. In certain embodiments, the presently disclosed anti-α5 integrin antibody or antigen-binding fragment thereof does not bind to a mouse α5 integrin.

In certain embodiments, the mouse α5 integrin comprises a wild-type mouse α5 integrin or a fragment thereof. In certain embodiments, the wild-type mouse α5 integrin comprises the amino acid sequence with a Uniprot Reference No: P11688 (SEQ ID NO: 2). SEQ ID NO: 2 is provided below.

(SEQ ID NO: 2)
MGSWTPRSPRSPLHAVLLRWGPRRLPPLLPLLLLLWPPPLQVGGFNLDA
EAPAVLSGPPGSLFGFSVEFYRPGRDGVSVLVGAPKANTSQPGVLQGGA
VYVCPWGTSPIQCTTIQFDSKGSRILESSLYSAKGEEPVEYKSLQWFGA
TVRAHGSSILACAPLYSWRTEKDPQNDPVGTCYLSTENFTRILEYAPCR
SDFGSAAGQGYCQGGFSAEFTKTGRVVLGGPGSYFWQGQILSATQEQIS
ESYYPEYLINPVQGQLQTRQASSVYDDSYLGYSVAVGEFSGDDTEDFVA
GVPKGNLTYGYVTVLNGSDIHSLYNVSGEQMASYFGYAVAATDTNGDGL
DDLLVGAPLLMERTADGRPQEVGRVYIYLQRPAGIDPTPTLTLTGQDEF
SRFGSSLTPLGDLDQDGYNDVAIGAPFGGEAQQGVVFIFPGGPGGLSTK
PSQVLQPLWAAGRTPDFFGSALRGGRDLDGNGYPDLIVGSFGVDKALVY
RGRPIISASASLTIFPSMFNPEERSCSLEGNPVSCINLSFCLNASGKHV
PNSIGFEVELQLDWQKQKGGVRRALFLTSKQATLTQTLLIQNGAREDCR
EMKIYLRNESEFRDKLSPIHIALNFSLDPKAPMDSHGLRPVLHYQSKSR
IEDKAQILLDCGEDNICVPDLQLDVYGEKKHVYLGDKNALNLTFHAQNL
GEGGAYEAELRVTAPLEAEYSGLVRHPGNFSSLSCDYFAVNQSRQLVCD
LGNPMKAGTSLWGGLRFTVPHLQDTKKTIQFDFQILSKNLNNSQSNVVS
FPLSVEAQAQVSLNGVSKPEAVIFPVSDWNPQDQPQKEEDLGPAVHHVY
ELINQGPSSISQGVLELSCPQALEGQQLLYVTKVTGLSNCTSNYTPNSQ
GLELDPETSPHHLQKREAPGRSSTASGTQVLKCPEAKCFRLRCEFGPLH
RQESRSLQLHFRVWAKTFLQREYQPFSLQCEAVYEALKMPYQILPRQLP
QKKLQVATAVQWTKAEGSNGVPLWIIILAILFGLLLLGLLIYVLYKLGF
FKRSLPYGTAMEKAQLKPPATSDA

Other related α5 integrin polypeptides that are also encompassed by the term α5 integrin include fragments, derivatives (e.g., substitution, deletion, truncations, and insertion variants), fusion polypeptides, and interspecies homologs that retain α5 integrin activity and/or are sufficient to generate an anti-α5 integrin immune response. As those skilled in the art will appreciate, an anti-α5 integrin antibody or antigen-binding fragment thereof described herein can bind to an α5 integrin polypeptide, an α5 integrin polypeptide fragment, an α5 integrin antigen, and/or an α5 integrin epitope. An epitope may be part of a larger α5 integrin antigen, which may be part of a larger α5 integrin polypeptide fragment, which, in turn, may be part of a larger α5 integrin polypeptide. An α5 integrin may exist in a native or denatured form. An α5 integrin polypeptide described herein may be isolated from a variety of sources, such as from human tissue types or from another source, or prepared by recombinant or synthetic methods. An α5 integrin polypeptide may comprise a polypeptide having the same amino acid sequence as a corresponding α5 integrin polypeptide derived from nature. Orthologs to the α5 integrin polypeptide are also well known in the art.

In certain embodiments, the presently disclosed anti-α5 integrin antibody or antigen-binding fragment thereof does not bind to a β1 integrin (e.g., the β1 integrin subunit of an α1β1 integrin). B1 integrin is also known as B1 integrin, integrin beta-1, ITGB1 protein, CD29 antigen, fibronectin receptor subunit beta, and Glycoprotein Iia. The term “β1 integrin” refers to a polypeptide or a protein of any native β1 integrin from any vertebrate source, including mammals such as primates (e.g., humans, cynomolgus monkey (cyno), dogs, and rodents (e.g., mice and rats). The term “β1 integrin” encompasses “full-length,” unprocessed β1 integrin, as well as any form of β1 integrin or any fragment thereof that results from processing in the cell. The term β1 integrin also encompasses naturally occurring variants of β1 integrin, such as SNP variants, splice variants and allelic variants.

In certain embodiments, the presently disclosed anti-α5 integrin antibody or antigen-binding fragment thereof does not bind to human β1 integrin. In certain embodiments, the human β1 subunit comprises a wild-type human β1 integrin or a fragment thereof. In certain embodiments, the wild-type human β1 integrin comprises the amino acid sequence with a Uniprot Reference No: P05556-1 (SEQ ID NO: 3). SEQ ID NO: 3 is provided below.

(SEQ ID NO: 3)
MNLQPIFWIGLISSVCCVFAQTDENRCLKANAKSCGECIQAGPNCGWCT
NSTFLQEGMPTSARCDDLEALKKKGCPPDDIENPRGSKDIKKNKNVTNR
SKGTAEKLKPEDITQIQPQQLVLRLRSGEPQTFTLKFKRAEDYPIDLYY
LMDLSYSMKDDLENVKSLGTDLMNEMRRITSDFRIGFGSFVEKTVMPYI
STTPAKLRNPCTSEQNCTSPFSYKNVLSLINKGEVENELVGKQRISGNL
DSPEGGFDAIMQVAVCGSLIGWRNVTRLLVFSTDAGFHFAGDGKLGGIV
LPNDGQCHLENNMYTMSHYYDYPSIAHLVQKLSENNIQTIFAVTEEFQP
VYKELKNLIPKSAVGTLSANSSNVIQLIIDAYNSLSSEVILENGKLSEG
VTISYKSYCKNGVNGTGENGRKCSNISIGDEVQFEISITSNKCPKKDSD
SFKIRPLGFTEEVEVILQYICECECQSEGIPESPKCHEGNGTFECGACR
CNEGRVGRHCECSTDEVNSEDMDAYCRKENSSEICSNNGECVCGQCVCR
KRDNTNEIYSGKFCECDNFNCDRSNGLICGGNGVCKCRVCECNPNYTGS
SVDCSLDTSTCEASNGQICNGRGICECGVCKCTDPKFQGQTCEMCQTCL
GVCAEHKECVQCRAFNKGEKKDTCTQECSYFNITKVESRDKLPQPVQPD
PVSHCKEKDVDDCWFYFTYSVNGNNEVMVHVVENPECPTGPDIIPIVAG
VVAGIVLIGLALLLIWKLLMIIHDRREFAKFEKEKMNAKWDTGENPIYK
SAVTTVVNPKYEGK

In certain embodiments, the presently disclosed anti-α5 integrin antibody or antigen-binding fragment thereof does not bind to a mouse β1 integrin. In certain embodiments, the mouse β1 subunit comprises a wild-type mouse β1 integrin or a fragment thereof. In certain embodiments, the mouse β1 integrin comprises the amino acid sequence with a Uniprot Reference No: P09055-1 (SEQ ID NO: 4). SEQ ID NO: 4 is provided below.

(SEQ ID NO: 4)
MNLQLVSWIGLISLICSVFGQTDKNRCLKANAKSCGECIQAGPNCGWCT
NTTFLQEGMPTSARCDDLEALKKKGCQPSDIENPRGSQTIKKNKNVTNR
SKGMAEKLRPEDITQIQPQQLLLKLRSGEPQKFTLKFKRAEDYPIDLYY
LMDLSYSMKDDLENVKSLGTDLMNEMRRITSDFRIGFGSFVEKTVMPYI
STTPAKLRNPCTSEQNCTSPFSYKNVLSLTDRGEFFNELVGQQRISGNL
DSPEGGFDAIMQVAVCGSLIGWRNVTRLLVFSTDAGFHFAGDGKLGGIV
LPNDGQCHLENNVYTMSHYYDYPSIAHLVQKLSENNIQTIFAVTEEFQP
VYKELKNLIPKSAVGTLSGNSSNVIQLIIDAYNSLSSEVILENSKLPDG
VTINYKSYCKNGVNGTGENGRKCSNISIGDEVQFEISITANKCPNKESE
TIKIKPLGFTEEVEVVLQFICKCNCQSHGIPASPKCHEGNGTFECGACR
CNEGRVGRHCECSTDEVNSEDMDAYCRKENSSEICSNNGECVCGQCVCR
KRDNTNEIYSGKFCECDNFNCDRSNGLICGGNGVCRCRVCECYPNYTGS
ACDCSLDTGPCLASNGQICNGRGICECGACKCTDPKFQGPTCETCQTCL
GVCAEHKECVQCRAFNKGEKKDTCAQECSHFNLTKVESREKLPQPVQVD
PVTHCKEKDIDDCWFYFTYSVNGNNEAIVHVVETPDCPTGPDIIPIVAG
VVAGIVLIGLALLLIWKLLMIIHDRREFAKFEKEKMNAKWDTGENPIYK
SAVTTVVNPKYEGK

In certain embodiments, the presently disclosed anti-α5 integrin antibody or antigen-binding fragment thereof is capable of blocking or inhibiting the binding of α5β1 (e.g., human α5β1) to fibronectin (e.g., human fibronectin). The term “fibronectin” refers to a polypeptide or a protein of any native fibronectin from any vertebrate source, including mammals such as primates (e.g., humans, cynomolgus monkey (cyno)), dogs, and rodents (e.g., mice and rats). Fibronectin (FN) exists as a dimer or multimer linked through disulfide bonds and has a multi-modular structure composed predominantly of three different repeats termed FN-I, FN-II, and FN-III. In dimeric form, each of the two fibronectin subunits consists of 12 FN-I, 2 FN-II, and 15 to 17 FN-III modules, respectively. The term “fibronectin” also encompasses naturally occurring variants of fibronectin, such as SNP variants, splice variants and allelic variants. Fibronectin is an essential component of the extracellular matrix and has multiple protein-binding domains, including domains for fibrin-binding, collagen-binding, fibulin-1-binding, heparin-binding and syndecan-binding. Fibronectin is known in the art to interact (e.g., via RGD) with integrins and is a ligand for α5β1 integrin, α8β1 integrin and αvβ3 integrin.

In certain embodiments, the presently disclosed anti-α5 integrin antibody or antigen-binding fragment thereof is capable of blocking or inhibiting the binding of human α5β1 to human fibronectin. In certain embodiments, the human fibronectin comprises a wild-type human fibronectin or a fragment thereof. In certain embodiments, the wild-type human fibronectin comprises the amino acid sequence with a Uniprot Reference No: P02751-15 (SEQ ID NO: 5). SEQ ID NO: 5 is provided below.

(SEQ ID NO: 5)
MLRGPGPGLLLLAVQCLGTAVPSTGASKSKRQAQQMVQPQSPVAVSQSK
PGCYDNGKHYQINQQWERTYLGNALVCTCYGGSRGFNCESKPEAEETCF
DKYTGNTYRVGDTYERPKDSMIWDCTCIGAGRGRISCTIANRCHEGGQS
YKIGDTWRRPHETGGYMLECVCLGNGKGEWTCKPIAEKCFDHAAGTSYV
VGETWEKPYQGWMMVDCTCLGEGSGRITCTSRNRCNDQDTRTSYRIGDT
WSKKDNRGNLLQCICTGNGRGEWKCERHTSVQTTSSGSGPFTDVRAAVY
QPQPHPQPPPYGHCVTDSGVVYSVGMQWLKTQGNKQMLCTCLGNGVSCQ
ETAVTQTYGGNSNGEPCVLPFTYNGRTFYSCTTEGRQDGHLWCSTTSNY
EQDQKYSFCTDHTVLVQTRGGNSNGALCHFPFLYNNHNYTDCTSEGRRD
NMKWCGTTQNYDADQKFGFCPMAAHEEICTTNEGVMYRIGDQWDKQHDM
GHMMRCTCVGNGRGEWTCIAYSQLRDQCIVDDITYNVNDTFHKRHEEGH
MLNCTCFGQGRGRWKCDPVDQCQDSETGTFYQIGDSWEKYVHGVRYQCY
CYGRGIGEWHCQPLQTYPSSSGPVEVFITETPSQPNSHPIQWNAPQPSH
ISKYILRWRPKNSVGRWKEATIPGHLNSYTIKGLKPGVVYEGQLISIQQ
YGHQEVTRFDFTTTSTSTPVTSNTVTGETTPFSPLVATSESVTEITASS
FVVSWVSASDTVSGFRVEYELSEEGDEPQYLDLPSTATSVNIPDLLPGR
KYIVNVYQISEDGEQSLILSTSQTTAPDAPPDTTVDQVDDTSIVVRWSR
PQAPITGYRIVYSPSVEGSSTELNLPETANSVTLSDLQPGVQYNITIYA
VEENQESTPVVIQQETTGTPRSDTVPSPRDLQFVEVTDVKVTIMWTPPE
SAVTGYRVDVIPVNLPGEHGQRLPISRNTFAEVTGLSPGVTYYFKVFAV
SHGRESKPLTAQQTTKLDAPTNLQFVNETDSTVLVRWTPPRAQITGYRL
TVGLTRRGQPRQYNVGPSVSKYPLRNLQPASEYTVSLVAIKGNQESPKA
TGVFTTLQPGSSIPPYNTEVTETTIVITWTPAPRIGFKLGVRPSQGGEA
PREVTSDSGSIVVSGLTPGVEYVYTIQVLRDGQERDAPIVNKVVTPLSP
PTNLHLEANPDTGVLTVSWERSTTPDITGYRITTTPTNGQQGNSLEEVV
HADQSSCTFDNLSPGLEYNVSVYTVKDDKESVPISDTIIPEVPQLTDLS
FVDITDSSIGLRWTPLNSSTIIGYRITVVAAGEGIPIFEDFVDSSVGYY
TVTGLEPGIDYDISVITLINGGESAPTTLTQQTAVPPPTDLRFTNIGPD
TMRVTWAPPPSIDLTNFLVRYSPVKNEEDVAELSISPSDNAVVLTNLLP
GTEYVVSVSSVYEQHESTPLRGRQKTGLDSPTGIDFSDITANSFTVHWI
APRATITGYRIRHHPEHFSGRPREDRVPHSRNSITLTNLTPGTEYVVSI
VALNGREESPLLIGQQSTVSDVPRDLEVVAATPTSLLISWDAPAVTVRY
YRITYGETGGNSPVQEFTVPGSKSTATISGLKPGVDYTITVYAVTGRGD
SPASSKPISINYRTEIDKPSQMQVTDVQDNSISVKWLPSSSPVTGYRVT
TTPKNGPGPTKTKTAGPDQTEMTIEGLQPTVEYVVSVYAQNPSGESQPL
VQTAVTNIDRPKGLAFTDVDVDSIKIAWESPQGQVSRYRVTYSSPEDGI
HELFPAPDGEEDTAELQGLRPGSEYTVSVVALHDDMESQPLIGTQSTAI
PAPTDLKFTQVTPTSLSAQWTPPNVQLTGYRVRVTPKEKTGPMKEINLA
PDSSSVVVSGLMVATKYEVSVYALKDTLTSRPAQGVVTTLENVSPPRRA
RVTDATETTITISWRTKTETITGFQVDAVPANGQTPIQRTIKPDVRSYT
ITGLQPGTDYKIYLYTLNDNARSSPVVIDASTAIDAPSNLRFLATTPNS
LLVSWQPPRARITGYIIKYEKPGSPPREVVPRPRPGVTEATITGLEPGT
EYTIYVIALKNNQKSEPLIGRKKTDELPQLVTLPHPNLHGPEILDVPST
VQKTPFVTHPGYDTGNGIQLPGTSGQQPSVGQQMIFEEHGFRRTTPPTT
ATPIRHRPRPYPPNVGEEIQIGHIPREDVDYHLYPHGPGLNPNASTGQE
ALSQTTISWAPFQDTSEYIISCHPVGTDEEPLQFRVPGTSTSATLTGLT
RGATYNVIVEALKDQQRHKVREEVVTVGNSVNEGLNQPTDDSCFDPYTV
SHYAVGDEWERMSESGFKLLCQCLGFGSGHFRCDSSRWCHDNGVNYKIG
EKWDRQGENGQMMSCTCLGNGKGEFKCDPHEATCYDDGKTYHVGEQWQK
EYLGAICSCTCFGGQRGWRCDNCRRPGGEPSPEGTTGQSYNQYSQRYHQ
RTNTNVNCPIECFMPLDVQADREDSRE

4.3. Anti-α5 integrin Antibody and Antigen-binding Fragments Thereof

The present disclosure provides anti-α5 integrin antibodies and antigen-binding fragments thereof. The antibodies can be polyclonal antibodies, monoclonal antibodies, humanized antibodies, human antibodies, multispecific antibodies, bispecific antibodies, or hetero-conjugate antibodies, as well as variants thereof having increased or decreased affinity or other properties. In certain embodiments, the presently disclosed antibodies are humanized antibodies.

In certain embodiments, the presently disclosed anti-α5 integrin antibody and antigen-binding fragment thereof binds to human α5 integrin. In certain embodiments, the presently disclosed anti-α5 integrin antibody and antigen-binding fragment thereof binds to the extracellular domain of human α5 integrin. In certain embodiments, the α5β1 integrin is a human α5β1 integrin. In certain embodiments, the presently disclosed anti-α5 integrin antibody and antigen-binding fragment thereof binds to a human α5 integrin comprising the amino acid sequence set forth in SEQ ID NO: 1 or a fragment thereof (e.g., amino acids 42 to 995 of SEQ ID NO: 1 or a fragment thereof). In certain embodiments, the presently disclosed anti-α5 integrin antibody and antigen-binding fragment thereof binds to an α5 integrin expressed on the surface of a mammalian (e.g., human) cell, e.g., an α5 integrin-expressing tumor cell. In certain embodiments, the presently disclosed presently anti-α5 integrin antibody and antigen-binding fragment thereof binds an α5 integrin extracellular epitope exposed on a cell, e.g., a tumor cell (e.g., an α5 integrin epitope).

### 4.3.1. Monospecifc Antibodies and Antigen-binding Fragments Thereof

In certain embodiments, the anti-α5 integrin antibody or antigen-binding fragment thereof comprises a heavy chain variable region (V_{H}) and a light chain variable region (V_{L}). In certain embodiments, the V_{H} comprises a CDR1 comprising the amino acid sequence set forth in SEQ ID NO: 6, SEQ ID NO: 13, SEQ ID NO: 18, SEQ ID NO: 19, SEQ ID NO: 24, or a conversation modification thereof, a CDR2 comprising the amino acid sequence set forth in SEQ ID NO: 7, SEQ ID NO: 14, SEQ ID NO: 20, SEQ ID NO: 25, SEQ ID NO: 30, or a conversation modification thereof, and a CDR3 comprising the amino acid sequence set forth in SEQ ID NO: 9, SEQ ID NO: 15, SEQ ID NO: 21, SEQ ID NO: 26, or a conversation modification thereof. SEQ ID Nos: 6, 7, 9, 13-15, 18-21, 24-26, and 30 are provided in Table 2.

In certain embodiments, the V_{H} comprises a CDR1 comprising the amino acid sequence set forth in SEQ ID NO: 6, SEQ ID NO: 13, SEQ ID NO: 18, SEQ ID NO: 19, SEQ ID NO: 24, or a conversation modification thereof, a CDR2 comprising the amino acid sequence set forth in SEQ ID NO: 8, SEQ ID NO: 14, SEQ ID NO: 20, SEQ ID NO: 25, SEQ ID NO: 30, or a conversation modification thereof, and a CDR3 comprising the amino acid sequence set forth in SEQ ID NO: 9, SEQ ID NO: 15, SEQ ID NO: 21, SEQ ID NO: 26, or a conversation modification thereof. SEQ ID Nos: 6, 8, 9, 13-15, 18-21, 24-26, and 30 are provided in Table 3.

In certain embodiments, the V_{L} comprises a CDR1 comprising the amino acid sequence set forth in SEQ ID NO: 10, SEQ ID NO: 16, SEQ ID NO: 22, SEQ ID NO: 27, or a conversation modification thereof, a CDR2 comprising the amino acid sequence set forth in SEQ ID NO: 11, SEQ ID NO: 17, SEQ ID NO: 28, or a conversation modification thereof, and a CDR3 comprising the amino acid sequence set forth in SEQ ID NO: 12, SEQ ID NO: 23, SEQ ID NO: 29, or a conversation modification thereof. SEQ ID NOs: 10-12, 16, 17, 22, 23, and 27-29 are disclosed in Table 4.

In certain embodiments, the presently disclosed anti-α5 integrin antibody or antigen-binding fragment thereof comprises a V_{H} comprising a CDR1 comprising the amino acid sequence set forth in SEQ ID NO: 13 or a conversation modification thereof, a CDR2 comprising the amino acid sequence set forth in SEQ ID NO: 14 or a conversation modification thereof, and a CDR3 comprising the amino acid sequence set forth in SEQ ID NO: 15 or a conversation modification thereof. In certain embodiments, the presently disclosed anti-α5 integrin antibody or antigen-binding fragment comprise a V_{L} comprising a CDR1 comprising the amino acid sequence set forth in SEQ ID NO: 16 or a conversation modification thereof, a CDR2 comprising the amino acid sequence set forth in SEQ ID NO: 17 or a conversation modification thereof, and a CDR3 comprising the amino acid sequence set forth in SEQ ID NO: 12 or a conversation modification thereof. In certain embodiments, the presently disclosed anti-α5 integrin antibody or antigen-binding fragment thereof comprises a V_{H} comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15. In certain embodiments, the presently disclosed anti-α5 integrin antibody or antigen-binding fragment comprise a V_{L} comprising a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 12. In certain embodiments, the presently disclosed anti-α5 integrin antibody or antigen-binding fragment thereof comprises a V_{H} comprising a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15; and a V_{L} comprising a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 12. In certain embodiments, the CDRs are identified according to the IMGT numbering system, e.g., as described in Lefranc, M.-P., 1999, The Immunologist, 7:132-136 and Lefranc, M.-P. et al., 1999, Nucleic Acids Res., 27:209-212.

In certain embodiments, the presently disclosed anti-α5 integrin antibody or antigen-binding fragment thereof comprises a V_{H} comprising a CDR1 comprising the amino acid sequence set forth in SEQ ID NO: 18 or a conversation modification thereof, a CDR2 comprising the amino acid sequence set forth in SEQ ID NO: 7 or a conversation modification thereof, and a CDR3 comprising the amino acid sequence set forth in SEQ ID NO: 9 or a conversation modification thereof. In certain embodiments, the presently disclosed anti-α5 integrin antibody or antigen-binding fragment comprise a V_{L} comprising a CDR1 comprising the amino acid sequence set forth in SEQ ID NO: 10 or a conversation modification thereof, a CDR2 comprising the amino acid sequence set forth in SEQ ID NO: 11 or a conversation modification thereof, and a CDR3 comprising the amino acid sequence set forth in SEQ ID NO: 12 or a conversation modification thereof. In certain embodiments, the presently disclosed anti-α5 integrin antibody or antigen-binding fragment thereof comprises a V_{H} comprising a CDR1 comprising the amino acid sequence set forth in SEQ ID NO: 18, a CDR2 comprising the amino acid sequence set forth in SEQ ID NO: 7, and a CDR3 comprising the amino acid sequence set forth in SEQ ID NO: 9. In certain embodiments, the presently disclosed anti-α5 integrin antibody or antigen-binding fragment comprise a V_{L} comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12. In certain embodiments, the presently disclosed anti-α5 integrin antibody or antigen-binding fragment thereof comprises a V_{H} comprising a CDR1 comprising the amino acid sequence set forth in SEQ ID NO: 18, a CDR2 comprising the amino acid sequence set forth in SEQ ID NO: 7, and a CDR3 comprising the amino acid sequence set forth in SEQ ID NO: 9; and a V_{L} comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12. In certain embodiments, the CDRs are identified according to the Kabat numbering system, e.g., as described in Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In certain embodiments, the presently disclosed anti-α5 integrin antibody or antigen-binding fragment thereof comprises a V_{H} comprising a CDR1 comprising the amino acid sequence set forth in SEQ ID NO: 18 or a conversation modification thereof, a CDR2 comprising the amino acid sequence set forth in SEQ ID NO: 8 or a conversation modification thereof, and a CDR3 comprising the amino acid sequence set forth in SEQ ID NO: 9 or a conversation modification thereof. In certain embodiments, the presently disclosed anti-α5 integrin antibody or antigen-binding fragment comprise a V_{L} comprising a CDR1 comprising the amino acid sequence set forth in SEQ ID NO: 10 or a conversation modification thereof, a CDR2 comprising the amino acid sequence set forth in SEQ ID NO: 11 or a conversation modification thereof, and a CDR3 comprising the amino acid sequence set forth in SEQ ID NO: 12 or a conversation modification thereof. In certain embodiments, the presently disclosed anti-α5 integrin antibody or antigen-binding fragment thereof comprises a V_{H} comprising a CDR1 comprising the amino acid sequence set forth in SEQ ID NO: 18, a CDR2 comprising the amino acid sequence set forth in SEQ ID NO: 8, and a CDR3 comprising the amino acid sequence set forth in SEQ ID NO: 9. In certain embodiments, the presently disclosed anti-α5 integrin antibody or antigen-binding fragment comprise a V_{L} comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12. In certain embodiments, the presently disclosed anti-α5 integrin antibody or antigen-binding fragment thereof comprises a V_{H} comprising a CDR1 comprising the amino acid sequence set forth in SEQ ID NO: 18, a CDR2 comprising the amino acid sequence set forth in SEQ ID NO: 8, and a CDR3 comprising the amino acid sequence set forth in SEQ ID NO: 9; and a V_{L} comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12. In certain embodiments, the CDRs are identified according to the Kabat numbering system.

In certain embodiments, the presently disclosed anti-α5 integrin antibody or antigen-binding fragment thereof comprises a V_{H} comprising a CDR1 comprising the amino acid sequence set forth in SEQ ID NO: 19 or a conversation modification thereof, a CDR2 comprising the amino acid sequence set forth in SEQ ID NO: 20 or a conversation modification thereof, and a CDR3 comprising the amino acid sequence set forth in SEQ ID NO: 21 or a conversation modification thereof. In certain embodiments, the presently disclosed anti-α5 integrin antibody or antigen-binding fragment comprise a V_{L} comprising a CDR1 comprising the amino acid sequence set forth in SEQ ID NO: 22 or a conversation modification thereof, a CDR2 comprising the amino acid sequence set forth in SEQ ID NO: 17 or a conversation modification thereof, and a CDR3 comprising the amino acid sequence set forth in SEQ ID NO: 23 or a conversation modification thereof. In certain embodiments, the presently disclosed anti-α5 integrin antibody or antigen-binding fragment thereof comprises a V_{H} comprising a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21. In certain embodiments, the presently disclosed anti-α5 integrin antibody or antigen-binding fragment comprise a V_{L} comprising a CDR1 comprising the amino acid sequence set forth in SEQ ID NO: 22, a CDR2 comprising the amino acid sequence set forth in SEQ ID NO: 17, and a CDR3 comprising the amino acid sequence set forth in SEQ ID NO: 23. In certain embodiments, the presently disclosed anti-α5 integrin antibody or antigen-binding fragment thereof comprises a V_{H} comprising a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21; and a V_{L} comprising a CDR1 comprising the amino acid sequence set forth in SEQ ID NO: 22, a CDR2 comprising the amino acid sequence set forth in SEQ ID NO: 17, and a CDR3 comprising the amino acid sequence set forth in SEQ ID NO: 23. In certain embodiments, the CDRs are identified according to the Chothia system, e.g., as described in Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215 (1): 175-82; and U.S. Pat. No. 7,709,226.

In certain embodiments, the presently disclosed anti-α5 integrin antibody or antigen-binding fragment thereof comprises a V_{H} comprising a CDR1 comprising the amino acid sequence set forth in SEQ ID NO: 24 or a conversation modification thereof, a CDR2 comprising the amino acid sequence set forth in SEQ ID NO: 25 or a conversation modification thereof, and a CDR3 comprising the amino acid sequence set forth in SEQ ID NO: 26 or a conversation modification thereof. In certain embodiments, the presently disclosed anti-α5 integrin antibody or antigen-binding fragment comprise a V_{L} comprising a CDR1 comprising the amino acid sequence set forth in SEQ ID NO: 27 or a conversation modification thereof, a CDR2 comprising the amino acid sequence set forth in SEQ ID NO: 28 or a conversation modification thereof, and a CDR3 comprising the amino acid sequence set forth in SEQ ID NO: 29 or a conversation modification thereof. In certain embodiments, the presently disclosed anti-α5 integrin antibody or antigen-binding fragment thereof comprises a V_{H} comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26. In certain embodiments, the presently disclosed anti-α5 integrin antibody or antigen-binding fragment comprise a V_{L} comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29. In certain embodiments, the presently disclosed anti-α5 integrin antibody or antigen-binding fragment thereof comprises a V_{H} comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; and a V_{L} comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29. In certain embodiments, the CDRs are identified according to the Contact system, e.g., as described in MacCallum R M et al., 1996, J Mol Biol 5:732-745). The Contact CDRs are based on an analysis of the available complex crystal structures.

In certain embodiments, the presently disclosed anti-α5 integrin antibody or antigen-binding fragment thereof comprises a V_{H} comprising a CDR1 comprising the amino acid sequence set forth in SEQ ID NO: 6 or a conversation modification thereof, a CDR2 comprising the amino acid sequence set forth in SEQ ID NO: 30 or a conversation modification thereof, and a CDR3 comprising the amino acid sequence set forth in SEQ ID NO: 9 or a conversation modification thereof. In certain embodiments, the presently disclosed anti-α5 integrin antibody or antigen-binding fragment comprise a V_{L} comprising a CDR1 comprising the amino acid sequence set forth in SEQ ID NO: 10 or a conversation modification thereof, a CDR2 comprising the amino acid sequence set forth in SEQ ID NO: 11 or a conversation modification thereof, and a CDR3 comprising the amino acid sequence set forth in SEQ ID NO: 12 or a conversation modification thereof. In certain embodiments, the presently disclosed anti-α5 integrin antibody or antigen-binding fragment thereof comprises a V_{H} comprising a CDR1 comprising the amino acid sequence set forth in SEQ ID NO: 6, a CDR2 comprising the amino acid sequence set forth in SEQ ID NO: 30, and a CDR3 comprising the amino acid sequence set forth in SEQ ID NO: 9. In certain embodiments, the presently disclosed anti-α5 integrin antibody or antigen-binding fragment comprise a V_{L} comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12. In certain embodiments, the presently disclosed anti-α5 integrin antibody or antigen-binding fragment thereof comprises a V_{H} comprising a CDR1 comprising the amino acid sequence set forth in SEQ ID NO: 6, a CDR2 comprising the amino acid sequence set forth in SEQ ID NO: 30, and a CDR3 comprising the amino acid sequence set forth in SEQ ID NO: 9; and a V_{L} comprising a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12. In certain embodiments, the CDRs are identified according to the AbM system, e.g., as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001).

TABLE 2
VH sequences of A2-M5-Low + moderate

| | | Exemplary | IMGT | Kabat | Chothia | Contact | AbM |
|---|---|---|---|---|---|---|---|
| V_{H} | V_{H} | GYTFTIYWIN | GYTFTIYW | IYWIN | GYTFTIY | TIYWIN | GYTFTIYWIN |
| CDR | CDR1 | (SEQ ID NO: 6) | (SEQ ID | (SEQ ID NO: 18) | (SEQ ID | (SEQ ID | (SEQ ID NO: 6) |
| | | | NO: 13) | | NO: 19) | NO: 24) | |
| Seq. | V_{H} | KIYPGSISTDYNEKFKG | IYPGSIST | KIYPGSISTDYNEKFKG | PGSI | WIGKIYPGSISTD | KIYPGSISTD |
| | CDR2 | (SEQ ID NO: 7) | (SEQ ID | (SEQ ID NO: 7) | (SEQ ID | (SEQ ID | (SEQ ID NO: 30) |
| | | | NO: 14) | | NO: 20) | NO: 25) | |
| | V_{H} | TGTGGLAY | AITGTGGLAY | TGTGGLAY | GTGGLA | AITGTGGLA | TGTGGLAY |
| | CDR3 | (SEQ ID NO: 9) | (SEQ ID | (SEQ ID NO: 9) | (SEQ ID | (SEQ ID | (SEQ ID NO: 9) |
| | | | NO: 15) | | NO: 21) | NO: 26) | |

| | |
|---|---|
| V_{H} | QVQLVQPGAEVKKPGASVKLSCKASGYTFTIYWINWVRQAPGQGLEWIGKUYPGSISTDYNQKFQGRATLTVDTSTSTAYMELSSLRS |
| Seq. | EDTAVYYCAITGTGGLAYWGQGTLVTVSS (SEQ ID NO: 32) |

TABLE 3
V_{H} sequences of A2-M5-Low+moderate

| | | Exemplary | IMGT | Kabat | Chothia | Contact | AbM |
|---|---|---|---|---|---|---|---|
| V_{H} | V_{H} | GYTFTIYWIN | GYTFTIYW | IYWIN | GYTFTIY | TIYWIN | GYTFTIYWIN |
| CDR | CDR1 | (SEQ ID | (SEQ ID | (SEQ ID | (SEQ ID | (SEQ ID | (SEQ ID |
| Seq. | | NO: 6) | NO: 13) | NO: 18) | NO: 19) | NO: 24) | NO: 6) |
| | V_{H} | KIYPGSISTDYNQ | IYPGSIST | KIYPGSISTDYNQ | PGSI | WIGKIYPGSI | KIYPGSISTD |
| | CDR2 | KFQG (SEQ ID | (SEQ ID | KFQG (SEQ ID | (SEQ ID | STD (SEQ ID | (SEQ ID |
| | | NO: 8) | NO: 14) | NO: 8) | NO: 20) | NO: 25) | NO: 30) |
| | V_{H} | TGTGGLAY | AITGTGGLAY | TGTGGLAY | GTGGLA | AITGTGGLA | TGTGGLAY |
| | CDR3 | (SEQ ID | (SEQ ID | (SEQ ID | (SEQ ID | (SEQ ID | (SEQ ID |
| | | NO: 9) | NO: 15) | NO: 9) | NO: 21) | NO: 26) | NO: 9) |

| | |
|---|---|
| V_{H} | QVQLVQPGAEVKKPGASVKLSCKASGYTFTIYWINWVRQAPGQGLEWIGKIYPGSISTDYNQKFQGRATLTVDTST |
| Seq. | STAYMELSSLRSEDTAVYYCAITGTGGLAYWGQGTLVTVSS (SEQ ID NO: 32) |

TABLE 4
V_{L} sequences of A2-M5-Low

| | | Exemplary | IMGT | Kabat | Chothia | Contact | AbM |
|---|---|---|---|---|---|---|---|
| V_{L} | V_{L} | RASSSVNYMY | SSVNY | RASSSVNYMY | SSSVNY | NYMYWY | RASSSVNYMY |
| CDR | CDR1 | (SEQ ID | (SEQ ID | (SEQ ID | (SEQ ID | (SEQ ID | (SEQ ID |
| Seq. | | NO: 10) | NO: 16) | NO: 10) | NO: 22) | NO: 27) | NO: 10) |
| | V_{L} | FTSSRAP | FTS | FTSSRAP | FTS | LWIYFTSSRA | FTSSRAP |
| | CDR2 | (SEQ ID | (SEQ ID | (SEQ ID | (SEQ ID | (SEQ ID | (SEQ ID |
| | | NO: 11) | NO: 17) | NO: 11) | NO: 17) | NO: 28) | NO: 11) |
| | V_{L} | QQFTTSPFT | QQFTTSPFT | QQFTTSPFT | FTTSPF | QQFTTSPF | QQFTTSPFT |
| | CDR3 | (SEQ ID | (SEQ ID | (SEQ ID | (SEQ ID | (SEQ ID | (SEQ ID |
| | | NO: 12) | NO: 12) | NO: 12) | NO: 23) | NO: 29) | NO: 12) |

| | |
|---|---|
| V_{L} | ENVLTQSPGIMSASLGERVTMSCRASSSVNYMYWYQQKSGQSPKLWIYFTSSRAPGVPDRFS |
| Seq. | GSGSGTDYTLTISRMEGEDFATYYCQQFTTSPFTFGQGTKLEIK (SEQ ID NO: 33) |

TABLE 5
V_{L} sequences of A2-M5-Low+moderate

| | | Exemplary | IMGT | Kabat | Chothia | Contact | AbM |
|---|---|---|---|---|---|---|---|
| V_{L} | V_{L} | RASSSVNYMY | SSVNY | RASSSVNYMY | SSSVNY | NYMYWY | RASSSVNYMY |
| CDR | CDR1 | (SEQ ID | (SEQ ID | (SEQ ID NO: 10) | (SEQ ID | (SEQ ID | (SEQ ID |
| Seq. | | NO: 10) | NO: 16) | | NO: 22) | NO: 27) | NO: 10) |
| | VL | FTSSRAP | FTS | FTSSRAP | FTS | LWIYFTSSRA | FTSSRAP |
| | CDR2 | (SEQ ID | (SEQ ID | (SEQ ID NO: 11) | (SEQ ID | (SEQ ID | (SEQ ID |
| | | NO: 11) | NO: 17) | | NO: 17) | NO: 28) | NO: 11) |
| | VL | QQFTTSPFT | QQFTTSPFT | QQFTTSPFT | FTTSPF | QQFTTSPF | QQFTTSPFT |
| | CDR3 | (SEQ ID | (SEQ ID | (SEQ ID NO: 12) | (SEQ ID | (SEQ ID | (SEQ ID |
| | | NO: 12) | NO: 12) | | NO: 23) | NO: 29) | NO: 12) |

| | |
|---|---|
| V_{L} | ENVLTQSPGILSASLGERVTMSCRASSSVNYMYWYQQKSGQAPRLWIYFTSSRAPGVPDRFSGSGSGTDYTL |
| Seq. | TISRMEGEDFATYYCQQFTTSPFTFGQGTKLEIK (SEQ ID NO: 34) |

In certain embodiments, conservative sequence modifications include conservative amino acid substitutions that include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in certain embodiments, a predicted nonessential amino acid residue in an anti-α5 integrin antibody or antigen-binding fragment thereof is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In certain embodiments, the amino acid sequence modifications refer to at most 1, 2, 3, 4, 5, or 6 amino acid substitutions to the CDRs described in Table 2. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In certain embodiments, the conservative modification is outside of a CDR sequence.

In certain embodiments, the presently disclosed anti-α5 integrin antibody or antigen-binding fragment thereof comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 31. In certain embodiments, the presently disclosed anti-α5 integrin antibody or antigen-binding fragment thereof comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 33. SEQ ID NOs: 31 and 33 are disclosed in Tables 2 and 4. In certain embodiments, the presently disclosed anti-α5 integrin antibody or antigen-binding fragment thereof comprises a V_{H} comprising the amino acid sequence set forth in SEQ ID NO: 31. In certain embodiments, the presently disclosed anti-α5 integrin antibody or antigen-binding fragment thereof comprises a V_{L} comprising the amino acid sequence set forth in SEQ ID NO: 33. In certain embodiments, the presently disclosed anti-α5 integrin antibody or antigen-binding fragment thereof comprises a V_{H} comprising the amino acid sequence set forth in SEQ ID NO: 31, and a V_{L} comprising the amino acid sequence set forth in SEQ ID NO: 33.

In certain embodiments, the presently disclosed anti-α5 integrin antibody or antigen-binding fragment thereof comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 31. In certain embodiments, the presently disclosed anti-α5 integrin antibody or antigen-binding fragment thereof comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 34. SEQ ID NO: 34 is disclosed in Table 5. In certain embodiments, the presently disclosed anti-α5 integrin antibody or antigen-binding fragment thereof comprises a V_{H} comprising the amino acid sequence set forth in SEQ ID NO: 31. In certain embodiments, the presently disclosed anti-α5 integrin antibody or antigen-binding fragment thereof comprises a V_{L} comprising the amino acid sequence set forth in SEQ ID NO: 34. In certain embodiments, the presently disclosed anti-α5 integrin antibody or antigen-binding fragment thereof comprises a V_{H} comprising the amino acid sequence set forth in SEQ ID NO: 31, and a V_{L} comprising the amino acid sequence set forth in SEQ ID NO: 34.

In certain embodiments, the presently disclosed anti-α5 integrin antibody or antigen-binding fragment thereof comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 32. In certain embodiments, the presently disclosed anti-α5 integrin antibody or antigen-binding fragment thereof comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 33. SEQ ID NO: 32 is disclosed in Table 3. In certain embodiments, the presently disclosed anti-α5 integrin antibody or antigen-binding fragment thereof comprises a V_{H} comprising the amino acid sequence set forth in SEQ ID NO: 32. In certain embodiments, the presently disclosed anti-α5 integrin antibody or antigen-binding fragment thereof comprises a V_{L} comprising the amino acid sequence set forth in SEQ ID NO: 33. In certain embodiments, the presently disclosed anti-α5 integrin antibody or antigen-binding fragment thereof comprises a V_{H} comprising the amino acid sequence set forth in SEQ ID NO: 32, and a V_{L} comprising the amino acid sequence set forth in SEQ ID NO: 33.

In certain embodiments, the presently disclosed anti-α5 integrin antibody or antigen-binding fragment thereof comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 32. In certain embodiments, the presently disclosed anti-α5 integrin antibody or antigen-binding fragment thereof comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 34. In certain embodiments, the presently disclosed anti-α5 integrin antibody or antigen-binding fragment thereof comprises a V_{H} comprising the amino acid sequence set forth in SEQ ID NO: 32. In certain embodiments, the presently disclosed anti-α5 integrin antibody or antigen-binding fragment thereof comprises a V_{L} comprising the amino acid sequence set forth in SEQ ID NO: 34. In certain embodiments, the presently disclosed anti-α5 integrin antibody or antigen-binding fragment thereof comprises a V_{H} comprising the amino acid sequence set forth in SEQ ID NO: 32, and a V_{L} comprising the amino acid sequence set forth in SEQ ID NO: 34.

The antibody designated as “A2-M5-Low(H)/Low(L)” comprises the V_{H} CDR1, V_{H} CDR2, and V_{H} CDR3 sequences disclosed in Table 2; and the V_{L} CDR1, V_{L} CDR2, and V_{L} CDR3 sequences disclosed in Table 4. In certain embodiments, the A2-M5-Low(H)/Low(L) antibody comprises a V_{H} comprising the amino acid sequence set forth in SEQ ID NO: 31 (disclosed in Table 2) and a V_{L} comprising the amino acid sequence set forth in SEQ ID NO: 33 (disclosed in Table 4).

The antibody designated as “A2-M5-Low(H)/Low+Mod(L)” comprises the V_{H} CDR1, V_{H} CDR2, and V_{H} CDR3 sequences disclosed in Table 2; and the V_{L} CDR1, V_{L} CDR2, and V_{L} CDR3 sequences disclosed in Table 5. In certain embodiments, the A2-M5-Low(H)/Low+Mod(L) antibody comprises a V_{H} comprising the amino acid sequence set forth in SEQ ID NO: 31 (disclosed in Table 2) and a V_{L} comprising the amino acid sequence set forth in SEQ ID NO: 34 (disclosed in Table 5).

The antibody designated as “A2-M5-Low+Mod(H)/Low(L)” comprises the V_{H} CDR1, V_{H} CDR2, and V_{H} CDR3 sequences disclosed in Table 3; and the V_{L} CDR1, V_{L} CDR2, and V_{L} CDR3 sequences disclosed in Table 4. In certain embodiments, the A2-M5-Low+Mod(H)/Low(L) antibody comprises a V_{H} comprising the amino acid sequence set forth in SEQ ID NO: 32 (disclosed in Table 3) and a V_{L} comprising the amino acid sequence set forth in SEQ ID NO: 33 (disclosed in Table 4).

The antibody designated as “A2-M5-Low+Mod(H)/Low+Mod(L)” comprises the V_{H} CDR1, V_{H} CDR2, and V_{H} CDR3 sequences disclosed in Table 3; and the V_{L} CDR1, V_{L} CDR2, and V_{L} CDR3 sequences disclosed in Table 5. In certain embodiments, the A2-M5-Low+Mod(H)/Low+Mod(L) antibody comprises a V_{H} comprising the amino acid sequence set forth in SEQ ID NO: 32 (disclosed in Table 3) and a V_{L} comprising the amino acid sequence set forth in SEQ ID NO: 34 (disclosed in Table 5).

In certain embodiments, the presently disclosed anti-α5 integrin antibody comprises a fragment crystallizable region (Fc region). Fc region is the C-terminal region of an immunoglobulin heavy chain. The Fc region can be a functional Fc region, a native Fc region, a recombinant Fc region, or a variant Fc region.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding region or binding domain (e.g., an antibody variable region or domain) and can be assessed using various assays as disclosed.

A “native Fc region” encompasses a Fc region found in nature, and not manipulated, modified, and/or changed (e.g., isolated, purified, selected, including or combining with other sequences such as variable region sequences) by a human. Non-limiting examples of native human Fc regions include a native human IgG1 Fc region (non-A and A allotypes), a native human IgG2 Fc region, a native human IgG3 Fc region a native human IgG4 Fc region as well as naturally occurring variants of the foregoing.

A “variant Fc region” comprises an amino acid sequence which differs from that of a native sequence Fc region by virtue of at least one amino acid modification, (e.g., substituting, addition, or deletion) preferably one or more amino acid substitution(s). In certain embodiments, the variant Fc region comprises at least one amino acid substitution compared to a native Fc region or to the Fc region of a parent polypeptide, e.g., from about 1 to about 10 amino acid substitutions, from about 1 to about 5 amino acid substitutions in a native sequence Fc region or in the Fc region of the parent polypeptide. The variant Fc region described herein can possess at least about 80%, at least about 90%, at least about 95% or at least about 99% sequence identity with a native Fc region and/or with an Fc region of a parent polypeptide. A variant Fc region may comprises a loss of effector function (e.g., silent Fc). Non-limiting examples of variant Fc regions include the following variants (according to the EU numbering system): N297A/Q (N297A or N297Q), LALA (L234A, L235A), LALAPS (L234A, L235A, P331S), LALAPG (L234A, L235A, P329G), and TM (L234F, L235E, P331S).

In certain embodiments, the Fc region comprises a V_{H}, and one or more heavy chain constant domains (e.g., CH1, Hinge, CH2, and CH3). In certain embodiments, the heavy chain constant region is chosen from IgG1, IgG2, IgG3, IgG4, IgM, IgA1, IgA2, IgD, and IgE. In certain embodiments, the heavy chain constant region is chosen from IgG1, IgG2, IgG3, and IgG4. In certain embodiments, the immunoglobulin isotype is IgG4. In certain embodiments, the immunoglobulin isotype is human IgG4. In certain embodiments, the immunoglobulin isotype is IgG1. In certain embodiments, the immunoglobulin isotype is human IgG1.

In certain embodiments, the presently disclosed anti-α5 integrin antibody or antigen-binding fragment thereof comprises a human IgG1 Fc region. In certain embodiments, the human IgG1 Fc region is a native human IgG1 Fc region. In certain embodiments, the native human IgG1 Fc region comprises the amino acid sequence set forth in SEQ ID NO: 35, which is provided below. Amino acids 6 to 115 of SEQ ID NO: 35 is the CH2 domain, and amino acids 116 to 222 of SEQ ID NO: 35 is the CH3 domain.

(SEQ ID NO: 35)
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK

In certain embodiments, the presently disclosed anti-α5 integrin antibody or antigen-binding fragment thereof comprises a human IgG1 heavy chain constant domain. In certain embodiments, the human IgG1 heavy chain constant domain comprises a native human IgG1 Fc region. In certain embodiments, the human IgG1 heavy chain constant domain comprises a native human IgG1 Fc region consisting of the amino acid sequence set forth in SEQ ID NO: 35. In certain embodiments, the human IgG1 heavy chain constant domain comprises the amino acid sequence set forth in SEQ ID NO: 36, which is provided below.

(SEQ ID NO: 36)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTOKSLSLSPGK

In certain embodiments, the presently disclosed anti-α5 integrin antibody or antigen-binding fragment thereof comprises a human IgG4 Fc region. In certain embodiments, the human IgG4 Fc region is a native human IgG4 Fc region. In certain embodiments, the native human IgG4 Fc region comprises the amino acid sequence set forth in SEQ ID NO: 37, which is provided below.

(SEQ ID NO: 37)
CPSCPAPEFLGGPSVFLFPPKPKDTLMISRTPEVTCVVVDVSQEDPEVQ
FNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGN
VFSCSVMHEALHNHYTQKSLSLSLGK

In certain embodiments, the presently disclosed anti-α5 integrin antibody or antigen-binding fragment thereof comprises a variant human IgG1 Fc region. In certain embodiments, the variant human IgG1 Fc region is a silent Fc region. In certain embodiments, the variant human IgG1 Fc region comprises two L→A substitutions in the CH2 domain. In certain embodiments, the variant human IgG1 Fc region comprises the amino acid sequence set forth in SEQ ID NO: 38. SEQ ID NO: 38 is provided below. In certain embodiments, the CH2 domain comprises amino acids 6 to 115 of SEQ ID NO: 38. In certain embodiments, the CH3 domain comprises amino acids 116 to 222 of SEQ ID NO: 38.

(SEQ ID NO: 38)
CPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK

In certain embodiments, the presently disclosed anti-α5 integrin antibody or antigen-binding fragment thereof comprises a human IgG1 heavy chain constant domain. In certain embodiments, the human IgG1 heavy chain constant domain comprises a variant human IgG1 Fc region. In certain embodiments, the human IgG1 heavy chain constant domain comprises a variant human IgG1 Fc region consisting of the amino acid sequence set forth in SEQ ID NO: 38. In certain embodiments, the human IgG1 heavy chain constant domain comprises the amino acid sequence set forth in SEQ ID NO: 39, which is provided below.

(SEQ ID NO: 39)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In certain embodiments, the presently disclosed anti-α5 integrin antibody or antigen-binding fragment thereof comprises a variant human IgG1 Fc region. In certain embodiments, the variant human IgG1 Fc region comprises one N→Q substitution in the CH2 domain. In certain embodiments, the variant human IgG1 Fc region comprises the amino acid sequence set forth in SEQ ID NO: 40, which is provided below.

(SEQ ID NO: 40)
CPPCPAPELLGGPSVFLFPPKPKDTLMISRTPEVTCVVVDVSHEDPEVK
FNWYVDGVEVHNAKTKPREEQYASTYRVVSVLTVLHQDWLNGKEYKCKV
SNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTOKSLSLSPGK

In certain embodiments, the presently disclosed anti-α5 integrin antibody or antigen-binding fragment thereof comprises a variant human IgG1 Fc region. In certain embodiments, the variant human IgG1 Fc region comprises two L→A substitutions and one P→S substitution in the CH2 domain. In certain embodiments, the variant human IgG1 Fc region comprises the amino acid sequence set forth in SEQ ID NO: 41, which is provided below.

(SEQ ID NO: 41)
CPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPASIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK

In certain embodiments, the presently disclosed anti-α5 integrin antibody or antigen-binding fragment thereof comprises a variant human IgG1 Fc region. In certain embodiments, the variant human IgG1 Fc region comprises two L→A substitutions and one P→G substitution in the CH2 domain. In certain embodiments, the variant human IgG1 Fc region comprises the amino acid sequence set forth in SEQ ID NO: 42, which is provided below.

(SEQ ID NO: 42)
CPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALGAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK

In certain embodiments, the presently disclosed anti-α5 integrin antibody or antigen-binding fragment thereof comprises a variant human IgG1 Fc region. In certain embodiments, the variant human IgG1 Fc region comprises one L→F substitution, one L→E substitution, and one P→S substitution in the CH2 domain. In certain embodiments, the variant human IgG1 Fc region comprises the amino acid sequence set forth in SEQ ID NO: 43, which is provided below.

(SEQ ID NO: 43)
CPPCPAPEFEGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPASIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK

In certain embodiments, the presently disclosed anti-α5 integrin antibody or antigen-binding fragment thereof comprises a variant human IgG4 Fc region. In certain embodiments, the variant human IgG4 Fc region comprises one P→S substitution, one A→F substitution, and one A→L substitution in the CH2 domain. In certain embodiments, the variant human IgG4 Fc region comprises the amino acid sequence set forth in SEQ ID NO: 44, which is provided below.

(SEQ ID NO: 44)
CPPCPAPEAAGGPSVFLFPPKPKDTLMISRTPEVTCVVVDVSQEDPEVQ
FNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGN
VFSCSVMHEALHNHYTQKSLSLSLGK

In certain embodiments, the presently disclosed anti-α5 integrin antibody or antigen-binding fragment thereof comprises a human IgG4 heavy chain constant domain. In certain embodiments, the human IgG4 heavy chain constant domain comprises a variant human IgG4 Fc region. In certain embodiments, the human IgG4 heavy chain constant domain comprises a variant human IgG4 Fc region consisting of the amino acid sequence set forth in SEQ ID NO: 44. In certain embodiments, the human IgG4 heavy chain constant domain comprises the amino acid sequence set forth in SEQ ID NO: 45, which is provided below.

(SEQ ID NO: 45)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEGK

In certain embodiments, the presently disclosed anti-α5 integrin antibody or antigen-binding fragment thereof comprises a light chain constant domain. In certain embodiments, the light chain constant region is chosen from kappa and lambda. In certain embodiments, the light chain constant region is kappa. In certain embodiments, the light chain constant domain comprises the amino acid sequence set forth in SEQ ID NO: 46, which is provided below.

(SEQ ID NO: 46)
RTVAAPSVFIFPPSDSQLKSGTASVVCLLNNFYPREAKVOWKVDNALQS
GNSQESVTEQDSKDSTYSLSSTLTLSKADYEKHKVYACEVTHQGLSSPV
TKSFNRGEC

In certain embodiments, the presently disclosed anti-α5 integrin antibody is a humanized antibody. In certain embodiments, the presently disclosed anti-α5 integrin antibody is a monoclonal antibody. In certain embodiments, the presently disclosed anti-α5 integrin antibody is a humanized monoclonal antibody. In certain embodiments, the presently disclosed antigen-binding fragment is a Fab, Fab′, F(ab′)₂, Fv, or single chain variable fragment (scFv). In certain embodiments, a presently disclosed antigen-binding fragment is an scFv.

### 4.3.2. Multispecific Molecules

The present disclosure provides multispecific molecules comprising the anti-α5 integrin antibody or antigen-binding fragment thereof disclosed herein. In certain embodiments, the multispecific molecules are multispecific antibodies. In certain embodiments, the multispecific molecules (e.g., multispecific antibodies) are bispecific molecules (e.g., bispecific antibodies).

In certain embodiments, the multispecific molecule binds to at least two different binding sites or target molecules. In certain embodiments, the multispecific molecule comprises at least a first binding specificity for an α5 integrin or an α5β1 integrin, and a second binding specificity for a second target epitope. The second target epitope can be an α5 integrin epitope, or a non-α5 integrin epitope, e.g., a different second target antigen. Non-limiting examples of the second target antigen include αv integrin, β3 integrin, α4 integrin, β1 integrin, α4 integrin, β7 integrin, TREM2, TNFα, IL-6, IL-1B, CSF1, CSF-1R, C1Q, CD40L, FGFR, IL-12, and Type I interferons.

Methods for making multispecific molecules are known in the art, such as, by co-expression of two immunoglobulin heavy chain-light chain pairs, where the two heavy chains have different specificities. Exemplary structures of multispecific molecules are known in the art and are further described in Weidle et al., 2013, Cancer Genomics & Proteomics 10:1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, KA-body, orthogonal Fab.

In certain embodiments, BsIgG comprises heavy chains that are engineered for heterodimerization. For example, heavy chains can be engineered for heterodimerization using a “knobs-into-holes” strategy, a SEED platform, a common heavy chain (e.g., in KA-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific molecule format is IgG appended with an additional antigen-binding moiety. For example, monospecific IgG can be engineered to have bi-specificity by appending an additional antigen-binding unit onto the monospecific IgG, for example, at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG (DVD-Ig), IgG (H)-scFv, scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67 (2015): 95-106. In certain embodiments, an exemplary antibody format is a B-Body format for monospecific or multispecific (e.g., bispecific antibodies) as described in, for example, International Patent Application Publication No. WO 2018/075692 and US Patent Application Publication No. 2018/0118811.

Bispecific antibody fragments (BsAb) are a format of bispecific molecules that lack some or all of the antibody constant domains. For example, some BsAb lack an Fc region. In certain embodiments, bispecific antibody fragments include heavy and light chain regions that are connected by a peptide linker that permits efficient expression of the BsAb in a single host cell. Non-limiting examples of bispecific antibody fragments include, but are not limited to, nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example, bispecific fusion proteins can be linked to other proteins to add additional specificity and/or functionality. In certain embodiments, the dock-and-lock (DNL) method can be used to generate bispecific antibody molecules with higher valency. For example, bispecific antibody fusions to albumin binding proteins or human serum albumin can be extend the serum half-life of antibody fragments. In certain embodiments, chemical conjugation, for example, chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In certain embodiments, the conjugation improves the serum half-life.

Methods of production of multispecific molecules, including bispecific molecules, are known in the art. For example, multispecific molecules, including bispecific molecules, can be produced by separate expression of the component antibodies in different host cells and subsequent purification/assembly or by expression of the component antibodies in a single host cell. Purification of multispecific (e.g., bispecific) molecules can be performed by various methods known in the art, including affinity chromatography.

In certain embodiments, the anti-α5 integrin antibodies or antigen-binding fragments thereof disclosed herein can be provided in any antibody format disclosed herein or known in the art. In certain embodiments, the anti-α5 integrin antibodies or antigen-binding fragments thereof can be selected from Fabs-in-tandem-lg (FIT-1g); DVD-lg; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (BiTE; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; humanized bispecific IgG antibody (REGN1979) (Regeneron); Mab2 bispecific antibodies (F-Star); DVD-1g=dual variable domain immunoglobulin (AbbVie); kappa-lambda bodies; TBTI=tetravalent bispecific tandem Ig; and CrossMab (Roche).

In certain embodiments, the multispecific (e.g., bispecific) molecule comprises an α5 integrin binding domain that comprises a V_{H} sequence disclosed herein (e.g., one disclosed in Section 4.3.1, e.g., one disclosed in Tables 2 and 3). In certain embodiments, the α5 integrin binding domain comprises a V_{L} sequence disclosed herein (e.g., one disclosed in Section 4.3.1, e.g., one disclosed in Tables 4 and 5). In certain embodiments, the α5 integrin binding domain comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 31. In certain embodiments, the α5 integrin binding domain comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 33. In certain embodiments, the α5 integrin binding domain comprises a V_{H} comprising the amino acid sequence set forth in SEQ ID NO: 31. In certain embodiments, the α5 integrin binding domain comprises a V_{L} comprising the amino acid sequence set forth in SEQ ID NO: 33. In certain embodiments, the α5 integrin binding domain comprises a V_{H} comprising the amino acid sequence set forth in SEQ ID NO: 31, and a V_{L} comprising the amino acid sequence set forth in SEQ ID NO: 33.

In certain embodiments, the α5 integrin binding domain comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 31. In certain embodiments, the α5 integrin binding domain comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 34. In certain embodiments, the α5 integrin binding domain comprises a V_{H} comprising the amino acid sequence set forth in SEQ ID NO: 31. In certain embodiments, the α5 integrin binding domain comprises a V_{L} comprising the amino acid sequence set forth in SEQ ID NO: 34. In certain embodiments, the α5 integrin binding domain comprises a V_{H} comprising the amino acid sequence set forth in SEQ ID NO: 31, and a V_{L} comprising the amino acid sequence set forth in SEQ ID NO: 34.

In certain embodiments, the α5 integrin binding domain comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 32. In certain embodiments, the α5 integrin binding domain comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 33. In certain embodiments, the α5 integrin binding domain comprises a V_{H} comprising the amino acid sequence set forth in SEQ ID NO: 32. In certain embodiments, the α5 integrin binding domain comprises a V_{L} comprising the amino acid sequence set forth in SEQ ID NO: 33. In certain embodiments, the α5 integrin binding domain comprises a V_{H} comprising the amino acid sequence set forth in SEQ ID NO: 32, and a V_{L} comprising the amino acid sequence set forth in SEQ ID NO: 33.

In certain embodiments, the α5 integrin binding domain comprises a V_{H} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 32. In certain embodiments, the α5 integrin binding domain comprises a V_{L} comprising an amino acid sequence that is at least about 75%, at least about 80%, at least about 85%, at least about 90%, at least about 95%, at least about 96%, at least about 97%, at least about 98%, or at least about 99% identical to the amino acid sequence set forth in SEQ ID NO: 34. In certain embodiments, the α5 integrin binding domain comprises a V_{H} comprising the amino acid sequence set forth in SEQ ID NO: 32. In certain embodiments, the α5 integrin binding domain comprises a V_{L} comprising the amino acid sequence set forth in SEQ ID NO: 34. In certain embodiments, the α5 integrin binding domain n comprises a V_{H} comprising the amino acid sequence set forth in SEQ ID NO: 32, and a V_{L} comprising the amino acid sequence set forth in SEQ ID NO: 34.

In certain embodiments, the α5 integrin binding domain comprises a V_{H} comprising one or more CDRs, including three V_{H} CDRs, for example, V_{H} CDR1, V_{H} CDR2, and/or V_{H} CDR3 disclosed in Tables 2 and 3. In certain embodiments, the α5 integrin binding domain comprises a V_{L} comprising one or more CDRs, including three V_{L} CDRs, for example, V_{L} CDR1, V_{L} CDR2, and/or V_{L} CDR3 disclosed in Table 4.

### 4.3.3. Cross-competing Antibodies

The present disclosure further provides antibodies or antigen-binding fragments thereof that cross-compete for binding to an α5 integrin (e.g., a α5 integrin) with any of the presently disclosed anti-α5 integrin antibodies or antigen-binding fragments thereof (e.g., those disclosed in those disclosed in Section 4.3.1, e.g., A2-M5-Low(H)/Low(L), A2-M5-Low(H)/Low+Mod(L), A2-M5-Low+Mod(H)/Low(L), and A2-M5-Low+Mod(H)/Low+Mod(L). For example, the cross-competing antibodies bind to the same epitope region, e.g., same epitope, adjacent epitope, or overlapping as any of the presently disclosed anti-α5 integrin antibodies or antigen-binding fragments thereof (e.g., those disclosed in Section 4.3.1, e.g., A2-M5-Low(H)/Low(L), A2-M5-Low(H)/Low+Mod(L), A2-M5-Low+Mod(H)/Low(L), and A2-M5-Low+Mod(H)/Low+Mod(L)).

The competition can be determined by an assay in which the test antibody under study prevents or inhibits the specific binding of the reference antibody to a common epitope or a common antigen (e.g., α5 integrin). Numerous types of competitive binding assays can be used to determine if a test antibody competes with a reference antibody for binding to α5 integrin (e.g., human α5 integrin). Examples of assays that can be employed include solid phase direct or indirect radioimmunoassay (RIA), solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619) solid phase direct labeled assay, solid phase direct labeled sandwich assay (see, e.g., Harlow and Lane, (1988) Antibodies, A Laboratory Manual, Cold Spring Harbor Press); solid phase direct label RIA using 1-125 label (see, e.g., Morel et al., (1988) Molec. Immunol. 25:7-15); solid phase direct biotin-avidin EIA (see, e.g., Cheung, et al., (1990) Virology 176:546-552); and direct labeled RIA (Moldenhauer et al., (1990) Scand. J. Immunol. 32:77-82). Typically, such an assay involves the use of a purified antigen (e.g., α5 integrin, such as human α5 integrin) bound to a solid surface or cells bearing either of an un-labelled test antigen binding protein (e.g., test α5 integrin antibody) or a labeled reference antigen binding protein (e.g., reference anti-α5 integrin antibody). Competitive inhibition may be measured by determining the amount of label bound to the solid surface or cells in the presence of the test antigen binding protein. Usually the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common epitope or common antigen by at least about 20%, for example, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96% or at least about 97%, at least about 98%, or at least about 99% or more.

### 4.3.4. Conjugates

The present disclosure further provides conjugates comprising a presently disclosed anti-α5 integrin antibody or antigen-binding fragment thereof. In certain embodiments, the presently disclosed anti-α5 integrin antibody or antigen-binding fragment thereof is linked or conjugated (directly or indirectly) to a moiety with effector function, such as cytotoxic activity (e.g., a chemotherapeutic moiety or a radioisotope) or immune recruitment activity. In certain embodiments, the conjugate is an immunoconjugate. In certain embodiments, the conjugate is an antibody-drug conjugate (ADC). Moieties that are linked or conjugated (directly or indirectly) include drugs that are cytotoxic or non-cytotoxic. In certain embodiments, the presently disclosed anti-α5 integrin antibody or antigen-binding fragment thereof is linked or conjugated (directly or indirectly) to a moiety that facilitates isolation from a mixture (e.g., a tag) or a moiety with reporter activity (e.g., a detection label or reporter protein).

In certain embodiments, the presently disclosed anti-α5 integrin antibody or antigen-binding fragment thereof is conjugated or recombinantly linked (directly or indirectly) to a therapeutic agent (e.g., a cytotoxic agent). In certain embodiments, the presently disclosed anti-α5 integrin antibody or antigen-binding fragment thereof is conjugated or recombinantly linked (directly or indirectly) to a detectable agent (e.g., a labeled agent, including a labeled antibody). In certain embodiments, the presently disclosed anti-α5 integrin antibody or antigen-binding fragment thereof is conjugated or recombinantly linked (directly or indirectly) to a diagnostic agent. The conjugated or recombinantly linked antibodies can be useful, for example, for diagnosing or treating α5 integrin-mediated diseases, disorders, and conditions, including a cancer (e.g., a cancer associated with or characterized by tumor cells that express or overexpress α5 integrin), an angiogenesis-associated disease (e.g., a disease associated with or characterized by abnormal angiogenesis), and an inflammatory disease (e.g., a neuroinflammatory disease, including MS and ALS).

Such diagnosis and/or detection, including with a diagnostic agent and/or a detectable agent, can be accomplished, for example, by coupling a presently disclosed anti-α5 integrin antibody or antigen-binding fragment thereof to detectable substances (e.g., a labeled agent, including a labeled antibody) including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Labeled agents (e.g., a labeled antibody) which specifically bind to an anti-α5 integrin antibody or antigen-binding fragment thereof can be used for diagnostic purposes to detect, diagnose, or monitor an α5β1 integrin-mediated disease, disorder, or condition.

In certain embodiments, a presently disclosed anti-α5 integrin antibody or antigen-binding fragment thereof is recombinantly linked or conjugated (covalent or non-covalent conjugations, directly or indirectly) to a heterologous protein or polypeptide (or fragment thereof, for example, to a polypeptide (e.g., of about 10, about 20, about 30, about 40, about 50, about 60, about 70, about 80, about 90, or about 100 amino acids) to generate fusion proteins. The heterologous protein, polypeptide, or peptide to which the presently disclosed anti-α5 integrin antibody or antigen-binding fragment thereof is linked is useful for targeting an anti-α5 integrin antibody or antigen-binding fragment thereof to a particular cell (e.g., an α5β1 integrin-expressing cell, including a tumor cell). In certain embodiments, the fusion protein retains the biological activity of an anti-α5 integrin antibody or antigen-binding fragment thereof. Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of anti-α5 integrin antibodies or antigen-binding fragments thereof, including, for example, anti-α5 integrin antibodies or antigen-binding fragments thereof with higher affinities and lower dissociation rates. In certain embodiments, the anti-α5 integrin antibodies or antigen-binding fragments thereof may be altered by being subjected to random mutagenesis by error-prone PCR, random nucleotide insertion, or other methods prior to recombination. A nucleic acid encoding an anti-α5 integrin antibody or antigen-binding fragment thereof described herein may be recombined with one or more components, motifs, sections, parts, domains, fragments, etc. of one or more heterologous molecules.

In certain embodiments, the presently disclosed anti-α5 integrin antibody or antigen-binding fragment thereof is linked (directly or indirectly) to a marker or “tag” sequence, such as a peptide, to facilitate purification. In certain embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In certain embodiments, the presently disclosed anti-α5 integrin antibody or antigen-binding fragment thereof (e.g., an antibody, e.g., a humanized antibody) is attached to a solid support, which can be used for immunoassays or purification of a target antigen (e.g., α5 integrin or α5β1 integrin). Non-limiting examples of such solid supports include glass, cellulose, polyacrylamide, nylon, polystyrene, polyvinyl chloride, or polypropylene.

In certain embodiments, the presently disclosed anti-α5 integrin antibody or antigen-binding fragment thereof is linked or conjugated (directly or indirectly) to a second antibody to form an antibody hetero-conjugate.

The linker may be a “cleavable moiety” facilitating release of the linked or conjugated agent in a cell, but non-cleavable linkers are also contemplated herein. Linkers for use in conjugates (e.g., antibody-drug conjugates) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and an agent, including wherein the agent is a drug for the preparation of ADC, may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further provides conjugates of antibodies and agents, including wherein the agent is a drug for the preparation of ADC, may be prepared using any suitable methods as disclosed in the art (see, e.g., Bioconjugate Techniques (Hermanson ed., 2^{nd} ed. 2008)).

Conventional conjugation strategies for antibodies and agents, including wherein the agent is a drug for the preparation of ADC, have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen. In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids.

In certain embodiments, the presently disclosed anti-α5 integrin antibody or antigen-binding fragment thereof is conjugated to one or more cytotoxic agent(s) disclosed herein or known in the art in order to generate an ADC. In certain embodiments, the cytotoxic agent is a chemotherapeutic agent including, but not limited to, methotrexate, adriamycin, doxorubicin, melphalan, mitomycin C, chlorambucil, daunorubicin or other intercalating agents. In certain embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In certain embodiments, the cytotoxic agent is a radioisotope to produce a radioconjugate or a radioconjugated agent. A variety of radionuclides are available for the production of radioconjugated agents including, but not limited to, 90Y, 125I, 131I, 123I, 111In, 131In, 105Rh, 153Sm, 67Cu, 67Ga, 166Ho, 177Lu, 186Re, 188Re, and 212Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the presently disclosed anti-α5 integrin antibody or antigen-binding fragment thereof is conjugated to a drug, e.g., a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor. In certain embodiments, the mitotic inhibitor is a dolastatin, an auristatin, a maytansinoid, and a plant alkaloid. In certain embodiments, the drug is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In certain embodiments, the anti-tumor antibiotic is selected from the group consisting of an actinomycine, an anthracycline, a calicheamicin, and a duocarmycin. In certain embodiments, the actinomycine is a pyrrolobenzodiazepine (PBD).

### 4.3.5. Antibody Generation

The presently disclosed anti-α5 integrin antibodies or antigen-binding fragments thereof can be obtained by any suitable method, such as (but not limited to) immunization with whole tumor cells comprising α5 integrin and collection of antibodies, recombinant techniques, or screening libraries of antibodies or antibody fragments using α5 integrin extracellular domain epitopes. Monoclonal antibodies may be generated using a variety of known techniques (see, e.g., Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). For example, an exemplary technique for generating monoclonal antibodies comprises immunizing an animal with a human α5 integrin antigen and generating a hybridoma from spleen cells taken from the animal. A hybridoma may produce a monoclonal antibody or antibody fragment that binds to α5 integrin antigen.

In certain embodiments, the antibodies or antigen-binding fragments thereof can be isolated from antibody phage libraries, including as described herein. In certain embodiments, antibody phage libraries can be generated using the techniques described in, for example, Antibody Phage Display: Methods and Protocols, P. M. O'Brien and R. Aitken, eds, Humana Press, Totawa N.J., 2002. In certain embodiments, antibody clones can be selected by screening phage libraries. Phage libraries can contain phage that display various fragments of antibody variable region (Fv) fused to phage coat protein (e.g., Fab, scFv). Such phage libraries are screened for antibodies against the desired antigen. Clones expressing Fv fragments (e.g., Fab, scFv) capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which V_{H} and V_{L} are covalently linked through a short, flexible peptide, or as Fab fragments, in which they are each fused to a constant domain and interact non-covalently, as described, for example, in Winter et al., Ann. Rev. Immunol., 12:433-455 (1994).

Repertoires of V_{H} and V_{L} genes can be separately cloned by polymerase chain reaction (PCR) and recombined randomly in phage libraries, which can then be searched for antigen-binding clones as described, for example, in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described, for example, by Griffiths et al., EMBO J, 12:725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227:381-388 (1992).

Screening of the libraries can be accomplished by various techniques known in the art. For example, α5 integrin (e.g., an α5 integrin polypeptide, fragment or epitope) or α5β1 integrin (e.g., an α5β1 integrin polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8:309-314 (1990) and in WO 92/09690, and a low coating density of antigen as described in Marks et al., Biotechnol., 10:779-783 (1992).

A presently disclosed anti-α5 integrin antibody or antigen-binding fragment thereof can be obtained by designing a suitable antigen screening procedure to select for the phage clone of interest followed by construction of a full length anti-α5 integrin antibody clone using V_{H} and/or V_{L} sequences (e.g., the Fv sequences), or various CDR sequences from V_{H} and V_{L} sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

The present disclosure provides humanized antibodies that bind to α5 integrin (e.g., human α5 integrin). Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind to α5 integrin and/or α5β1 integrin may be produced using techniques known to those skilled in the art (e.g., Zhang et al., Molecular Immunology, 42 (12): 1445-1451, 2005; Hwang et al., Methods, 36 (1): 35-42, 2005; Dall'Acqua et al., Methods, 36 (1): 43-60, 2005; Clark, Immunology Today, 21 (8): 397-402, 2000, and U.S. Pat. Nos. 6,180,370; 6,054,927; 5,869,619; 5,861,155; 5,712,120; and 4,816,567.

In certain embodiments, the humanized antibodies are constructed by CDR grafting, in which the amino acid sequences of the six complementarity determining regions (CDRs) of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36:25-34, 2005).

The choice of human variable domains, both light and heavy, to be used in making the humanized antibodies, can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (see, e.g., Sims et al. (1993) J. Immunol. 151:2296; Chothia et al. (1987) J. Mol. Biol. 196:901. Another method uses a particular framework derived from the consensus sequences of all human antibodies of a particular subgroup of light or heavy chains. The same framework may be used for several different humanized antibodies (see, e.g., Carter et al. (1992) Proc. Natl. Acad. Sci. USA, 89:4285; Presta et al. (1993) J. Immunol., 151:2623. In some cases, the framework is derived from the consensus sequences of the most abundant human subclasses, V_{L}6 subgroup I (V_{L}6I) and V_{H} subgroup III (V_{H}III). In another method, human germline genes are used at the source of the framework regions.

In an alternative paradigm based on comparison of CDRs, called Superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169:1119-1125, 2002).

The antibodies are humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-824, 2000), Modeller (Sali and Blundell, J. Mol. Biol. 234:779-815, 1993), and Swiss PDB Viewer (Guex and Peitsch, Electrophoresis 18:2714-2713, 1997). Inspection of these displays permits analysis of the likely role of the residues in the functioning of the candidate immunoglobulin sequence, for example, the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. (see, e.g., Lazar et al., Mol. Immunol. 44:1986-1998, 2007).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23:1105-1116, 2005; Dufner et al., Trends Biotechnol. 24:523-529, 2006; Feldhaus et al., Nat. Biotechnol. 21:163-70, 2003; Schlapschy et al., Protein Eng. Des. Sel. 17:847-60, 2004).

In the FR library approach, a collection of residue variants are introduced at specific positions in the FR followed by selection of the library to select the FR that best supports the grafted CDR. The residues to be substituted may include some or all of the “Vernier” residues identified as potentially contributing to CDR structure (see, e.g., Foote and Winter, J. Mol. Biol. 224:487-499, 1992), or from the more limited set of target residues identified by Baca et al. (J. Biol. Chem. 272:10678-10684, 1997).

In FR shuffling, whole FRs are combined with the non-human CDRs instead of creating combinatorial libraries of selected residue variants (see, e.g., Dall'Acqua et al., Methods 36:43-60, 2005). The libraries may be screened for binding in a two-step selection process, first humanizing V_{L}, followed by V_{H}. Alternatively, a one-step FR shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44:3049-60, 2007).

The “humaneering” method is based on experimental identification of essential minimum specificity determinants (MSDs) and is based on sequential replacement of non-human fragments into libraries of human FRs and assessment of binding. It begins with regions of the CDR3 of non-human V_{H} and V_{L} chains and progressively replaces other regions of the non-human antibody into the human FRs, including the CDR1 and CDR2 of both V_{H} and V_{L}. This methodology typically results in epitope retention and identification of antibodies from multiple sub-classes with distinct human V-segment CDRs. Humaneering allows for isolation of antibodies that are 91-96% homologous to human germline gene antibodies. (see, e.g., Alfenito, Cambridge Healthtech Institute's Third Annual PEGS, The Protein Engineering Summit, 2007).

The “human engineering” method involves altering an non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low (“Low”) and/or moderate (“Mod”) risk positions in the non-human sequence can be substituted for the corresponding residues in the human antibody sequence according to the alignment. Techniques for making human engineered proteins are described in greater detail in Studnicka et al., Protein Engineering, 7:805-814 (1994), U.S. Pat. Nos. 5,766,886, 5,770,196, 5,821,123, and 5,869,619, and PCT Application Publication WO 93/11794.

### 4.4. Nucleic Acids Encoding the Antibodies and Vectors

The present disclosure provides nucleic acids encoding the anti-α5 integrin antibodies or antigen-binding fragments thereof disclosed herein. In certain embodiments, the nucleic acid comprises a first polynucleotide encoding an anti-α5 integrin V_{H} disclosed herein (e.g., one disclosed in Section 4.3.1, e.g., one disclosed in Tables 2 and 3). In certain embodiments, the first polynucleotide encodes an anti-α5 integrin V_{H} comprising the amino acid sequence set forth in SEQ ID NO: 31. In certain embodiments, the first polynucleotide encodes an anti-α5 integrin V_{H} comprising the amino acid sequence set forth in SEQ ID NO: 32. In certain embodiments, the first polynucleotide encodes an anti-α5 integrin V_{H} comprising a CDR1, a CDR2, and a CDR3 disclosed in Tables 2 and 3.

In certain embodiments, the nucleic acid comprises a second polynucleotide encoding an anti-α5 integrin V_{L} disclosed herein (e.g., one disclosed in Section 4.3.1, e.g., one disclosed in Tables 4 and 5). In certain embodiments, the second polynucleotide encodes an anti-α5 integrin V_{L} comprising the amino acid sequence set forth in SEQ ID NO: 33. In certain embodiments, the second polynucleotide encodes an anti-α5 integrin V_{L} comprising the amino acid sequence set forth in SEQ ID NO: 34. In certain embodiments, the second polynucleotide encodes an anti-α5 integrin V_{L} comprising a CDR1, a CDR2, and a CDR3 disclosed in Table 4.

Furthermore provided are vectors comprising the presently disclosed nucleic acids. In certain embodiments, the vector is an expression vector.

In certain embodiments, the presently disclosed nucleic acids are operatively linked to one or more polynucleotides comprising expression control sequences. Expression control sequences include promoters, enhancers, and operators, and are generally selected based on the expression systems in which the expression construct (e.g., expression vector) is to be utilized. Promoter and enhancer sequences are generally selected for the ability to increase gene expression, while operator sequences are generally selected for the ability to regulate gene expression. In certain embodiments, the expression vectors further comprise sequences encoding one or more selectable markers that permit identification of host cells bearing the construct. In certain embodiments, the expression vectors further comprise sequences that facilitate, and preferably promote, homologous recombination in a host cell. In certain embodiments, the expression vectors) further comprise sequences necessary for replication in a host cell.

Exemplary expression control sequences include promoter/enhancer sequences, including, for example, cytomegalovirus promoter/enhancer (Lehner et al., J. Clin. Microbiol., 29:2494-2502, 1991; Boshart et al., Cell, 41:521-530, 1985); Rous sarcoma virus promoter (Davis et al., Hum. Gene Ther., 4:151, 1993); Tie promoter (Korhonen et al., Blood, 86 (5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132:2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

In certain embodiments, the one or more polynucleotides comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. In certain embodiments, the one or more polynucleotides comprise sequences whose only intended function is to facilitate large scale production of the vector. One can manufacture and administer polynucleotides for gene therapy using procedures that have been described in the literature for a variety of transgenes. See, e.g., Isner et al., Circulation, 91:2687-2692, 1995; and Isner et al., Human Gene Therapy, 7:989-1011, 1996.

Any suitable vectors may be used to introduce the presently disclosed nucleic acids into the host cells. Exemplary vectors that have been described include replication deficient retroviral vectors, including but not limited to lentivirus vectors (see, e.g., Kim et al., J. Virol., 72 (1): 811-816, 1998; Kingsman & Johnson, Scrip Magazine, October 1998, pp. 43-46); parvoviral vectors, such as adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45:87-98, 1997); adenoviral (AV) vectors (see, e.g., U.S. Pat. Nos. 5,792,453; 5,824,544; 5,707,618; 5,693,509; 5,670,488; 5,585,362; Quantin et al., Proc. Natl. Acad. Sci. USA, 89:2581-2584, 1992; Stratford Perricaudet et al., J. Clin. Invest., 90:626-630, 1992; and Rosenfeld et al., Cell, 68:143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, for example, deleting or disrupting select genes required for viral replication.

Other non-viral delivery mechanisms contemplated include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985, DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am., 276 (6): 102-6, 1997; Felgner, Hum Gene Ther., 7 (15): 1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

An expression vector can be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814, 1997). Also contemplated are various commercial approaches involving “lipofection” technology. In certain embodiments, the liposome may be complexed with a hemagglutinating virus (HVJ). This has been shown to facilitate fusion with the cell membrane and promote cell entry of liposome-encapsulated DNA (see, e.g., Kaneda et al., Science, 243:375-378, 1989). In certain embodiments, the liposome is complexed or employed in conjunction with nuclear nonhistone chromosomal proteins (HMG-1) (see, e.g., Kato et al., J. Biol. Chem., 266:3361-3364, 1991). In certain embodiments, the liposomes are complexed or employed in conjunction with both HVJ and HMG-1. Such expression constructs have been successfully employed in transfer and expression of nucleic acid in vitro and in vivo. In certain embodiments, a presently disclosed anti-α5 integrin antibody or antigen-binding fragment thereof is included in the liposome to target the liposome to cells (such as tumor cells) expressing α5 integrin on their surface.

The present disclosure further provides host cells comprising the vectors disclosed herein. The host cells may be prokaryotic cells, such as Escherichia coli (see, e.g., Pluckthun et al., Methods Enzymol., 178:497-515, 1989), or eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., anti-α5 integrin antibodies or antigen-binding fragments thereof) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of an anti-α5 integrin antibody (e.g., a humanized antibody) may comprise culturing a host cell and isolating the anti-α5 integrin antibody. Transferring a naked DNA expression construct into cells can be accomplished using particle bombardment, which depends on the ability to accelerate DNA coated microprojectiles to a high velocity allowing them to pierce cell membranes and enter cells without killing them (see, e.g., Klein et al., Nature, 327:70-73, 1987). Several devices for accelerating small particles have been developed. One such device relies on a high voltage discharge to generate an electrical current, which in turn provides the motive force (see, e.g., Yang et al., Proc. Natl. Acad. Sci USA, 87:9568-9572, 1990). The microprojectiles used have consisted of biologically inert substances such as tungsten or gold beads. A host cell may be isolated and/or purified. A host cell also may be a cell transformed in vitro to cause transient or permanent expression of the polypeptide in vivo. A host cell may also be an isolated cell transformed ex vivo and introduced post-transformation, for example, to produce the polypeptide in vivo for therapeutic purposes. The definition of host cell explicitly excludes a transgenic human being.

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, UK, 1992); and Current Protocols in Molecular Biology, Ausubel ed., Wiley Interscience, New York, 1999).

In certain embodiments, an anti-α5 integrin antibody (e.g., a humanized antibody) is produced using any suitable method, for example, isolated from an immunized animal, recombinantly or synthetically generated, or genetically-engineered, including as described above. Antibody fragments derived from an antibody are obtained by, for example, proteolytic hydrolysis of an antibody. For example, papain or pepsin digestion of whole antibodies yields a 5S fragment termed F(ab′) 2 or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1:422 Academic Press (1967); Nisonoff et al., Arch. Biochem. Biophys., 89:230-244, 1960; Porter, Biochem. J., 73:119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1 2.8.10 and 2.10A.1 2.10A.5.

An anti-α5 integrin antibody (e.g., a humanized antibody) can be genetically engineered. For example, an anti-α5 integrin antibody (e.g., a humanized antibody) comprises, for example, a variable region or variable domain generated by recombinant DNA engineering techniques. In this regard, a variable region is optionally modified by insertions, deletions, or changes in the amino acid sequence of the antibody to produce an antibody of interest, including as described above. Polynucleotides encoding CDRs of interest, including CDRs as listed in Table 2, are prepared, for example, by using polymerase chain reaction to synthesize variable regions using mRNA of antibody producing cells as a template (see, e.g., Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2:106-110, 1991). Current antibody manipulation techniques allow construction of engineered variable region domains containing at least one CDR and, optionally, one or more framework amino acids from a first antibody and the remainder of the variable region domain from a second antibody. Such techniques are used, for example, to humanize an antibody or to improve its affinity for a binding target.

“Humanized antibodies” are antibodies in which CDRs of heavy and light variable chains of non-human immunoglobulins are transferred into a human variable domain. Constant regions need not be present, but if they are, they optionally are substantially identical to human immunoglobulin constant regions, for example, at least about 85-90%, at least about 95%, at least about 96%, at least about 97%, at least about 98%, at least about 99% or more identical, in certain embodiments. Hence, in some instances, all parts of a humanized immunoglobulin, except possibly the CDRs, are substantially identical to corresponding parts of natural human immunoglobulin sequences. For example, humanized antibodies are human immunoglobulins (e.g., host antibody) in which hypervariable region residues of the host antibody are replaced by hypervariable region residues from a non-human species (donor antibody) such as mouse, rat, rabbit, or a non-human primate having the desired specificity, affinity, and capacity.

### 4.5. Methods of Detection

The present disclosure provides methods for detecting α5 integrin in a whole cell or tissue. In certain embodiments, the method comprises: a) contacting a cell or tissue with an anti-α5 integrin antibody or antigen-binding fragment thereof disclosed herein, wherein the anti-α5 integrin antibody or antigen-binding fragment thereof comprises a detectable label; and b) determining the amount of the labeled anti-α5 integrin antibody or antigen-binding fragment thereof bound to the cell or tissue. In certain embodiments, the method is for detecting human α5 integrin.

In certain embodiments, b) determining the amount of the labeled anti-α5 integrin antibody or antigen-binding fragment thereof bound to the cell or tissue comprises measuring the amount of detectable label associated with the cell or tissue, wherein the amount of bound antibody or antigen-binding fragment thereof indicates the amount of α5 integrin (e.g., human α5 integrin) in the cell or tissue.

The cell or tissue can be any cell or tissue, including any normal, healthy, abnormal, tumor, or cancer cells and tissues.

The present disclosure also provides methods for detecting or diagnosing an α5 integrin-associated disease, disorder, or condition. A more definitive diagnosis of an α5 integrin-associated disease, disorder, or condition may allow health professionals to employ preventative measures or aggressive treatment earlier thereby preventing the development or further progression of the α5 integrin-associated disease, disorder, or condition. In certain embodiments, the method comprises: (a) measuring an expression level of α5 integrin in a cell or a tissue sample of a subject using an anti-α5 integrin antibody or antigen-binding fragment thereof disclosed herein; and (b) comparing the expression level of α5 integrin measured in (a) with a control expression level of α5 integrin, wherein an increase in the expression level of α5 integrin measured in (a) as compared to the control expression level of α5 integrin is indicative of an α5 integrin-associated disease, disorder, or condition. In certain embodiments, the control expression level of α5 integrin is an expression level of α5 integrin in a cell or a tissue sample of a subject not suffering from an α5 integrin-associated disease, disorder, or condition

### 4.6. Compositions and Formulations

The present disclosure provides compositions comprising the anti-α5 integrin antibodies or antigen-binding fragments thereof, the conjugates, or the multispecific molecules disclosed herein. In certain embodiments, the composition is a pharmaceutical composition further comprising a pharmaceutically acceptable carrier.

The particular carrier employed may depend on chemico-physical considerations, such as solubility and lack of reactivity with the anti-α5 integrin antibodies or antigen-binding fragments thereof or co-therapy, and by the route of administration. Pharmaceutically acceptable carriers are well-known in the art, examples of which are described herein. Illustrative pharmaceutical forms suitable for injectable use include sterile aqueous solutions or dispersions and sterile powders for the extemporaneous preparation of sterile injectable solutions or dispersions. Injectable formulations are further described in, for example, Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986)). In certain embodiments, a presently disclosed pharmaceutical composition is placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

In certain embodiments, the composition comprises a non-antibody protein scaffold. Non-limiting examples of non-antibody protein scaffolds include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate an anti-α5 integrin antibody comprising a non-antibody protein scaffold (see, e.g., Simeon and Chen, Protein Cell, 9 (1): 3-14 (2018); Yang et al., Annu Rev Anal Chem (Palo Alto Calif). 10(1):293-320 (2017)).

### 4.7. Methods of Treatment

The anti-α5 integrin antibodies, antigen-binding fragments thereof, multispecific molecules, conjugates and compositions can be used for treating an α5 integrin-associated disease, disorder, or condition, including one or more symptoms of the disease, disorder, or condition. Thus, the present disclosure provides methods of treating an α5 integrin-associated disease, disorder, or condition. In certain embodiments, the method comprises administering to the subject a presently disclosed anti-α5 integrin antibody, antigen-binding fragment thereof, multispecific molecule, conjugate, or composition. In certain embodiments, the subject suffers from or is diagnosed with an α5 integrin-associated disease, disorder, or condition. In certain embodiments, the subject is human.

In certain embodiments, the disease, disorder, or condition is associated with α5ß1. In certain embodiments, an α5β1 integrin-associated disease, disorder, or condition refers to any disease, disorder or condition that is completely or partially caused by or is the result of α5ß1 integrin or the interaction of α5β1 integrin with fibronectin and/or alternatively any disease, disorder, or condition in which it is desirable to inhibit the in vivo effects of the interaction of α5ß1 integrin with fibronectin. In certain embodiments, the disease, disorder, or condition is associated with over-expression of α5ß1. Non-limiting examples of α5 integrin-associated diseases, disorders, and conditions include tumors, angiogenesis-associated diseases (e.g., disease associated with or characterized by abnormal angiogenesis), and inflammatory diseases.

In certain embodiments, the α5β1 integrin-associated disease, disorder or condition is characterized by or associated with abnormally increased angiogenic activity of cells (e.g., tumor cells). In certain embodiments, the α5 integrin-associated disease, disorder or condition is a tumor. In certain embodiments, a cell of the tumor expresses or overexpresses an α5 integrin or an α5ß1 integrin. In certain embodiments, the tumor is a solid tumor. In certain embodiments, the tumor is cancer. In certain embodiments, the tumor or cancer is characterized by or associated with tumor or cancerous cells that express or over-express an α5 integrin or α5β1 integrin. Non-limiting examples of cancer include breast cancer, bladder cancer, melanoma, prostate cancer, mesothelioma, lung cancer, brain cancer, ovarian cancer, colon cancer, testicular cancer, thyroid cancer, squamous cell carcinoma, glioblastoma, neuroblastoma, neurofibromatosis, uterine cancer, colorectal cancer, stomach cancer, and pancreatic cancer.

The present disclosure also provides methods of modulating (e.g., inhibiting, reducing, preventing) tumor growth in a subject suffering from a tumor. For example, the method comprises administering to the subject a presently disclosed anti-α5 integrin antibody, antigen-binding fragment thereof, multispecific molecule, conjugate, or composition in an amount effective to modulate tumor growth in the subject. “Tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated or abnormal cell growth and includes all malignant neoplasms including, but not limited to: carcinoma, lymphoma, blastoma, sarcoma, and leukemia. Examples of cancers include, but are not limited to: breast cancer (including metastatic breast cancer), cervical cancer, colon cancer, colorectal cancer (including metastatic colorectal cancer), lung cancer (including non-small cell lung cancer), fibrosarcoma, non-Hodgkins lymphoma (NHL), chronic lymphocytic leukemia, bladder cancer, pancreatic cancer, renal cell cancer, spleen cancer, prostate cancer including hormone refractory prostate cancer, liver cancer, head and neck cancer, stomach cancer, bladder cancer, melanoma, ovarian cancer, mesothelioma, soft tissue cancer, gastrointestinal stromal tumor, glioblastoma multiforme and multiple myeloma.

“Inhibiting” does not require a 100% inhibition. Any inhibition that reduces tumor growth and/or metastasis is contemplated. Similarly, “modulating” tumor growth refers to reducing the size of the tumor, slowing tumor growth, or inhibiting an increase in the size of an existing tumor. Complete abolition of a tumor is not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size or tumor metastasis, a decrease or maintenance of the levels of tumor markers, or reduction or maintenance of a tumor cell population. In certain embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a presently disclosed anti-α5 integrin antibody, antigen-binding fragment thereof, multispecific molecule, conjugate, or composition.

In certain embodiments, an α5 integrin-associated disease, disorder or condition is associated with abnormal angiogenesis. In certain embodiments, the α5 integrin-associated disease, disorder or condition is an ocular disease. In certain embodiments, the ocular disease is selected from the group consisting of diabetic retinopathy, age-induced macular degeneration, and uveitis.

The present disclosure also provides methods of inhibiting abnormal angiogenesis in a subject. In certain embodiments, the subject suffers from a tumor. In certain embodiments, the method comprises administering to the subject an amount of an anti-α5 integrin antibody, antigen-binding fragment thereof, multispecific molecule, conjugate and composition described herein, effective to inhibit the abnormal angiogenesis.

In certain embodiments, the inflammatory disease is a neuroinflammatory disease. Non-limiting examples of inflammatory diseases include macrophages-mediated innate immunity diseases, multiple sclerosis, amyotrophic lateral sclerosis (ALS), atherosclerosis.

A particular administration regimen for a particular subject will depend, in part, upon the agent used, the amount of agent administered, the route of administration, and the cause and extent of any side effects. The amount of the anti-α5 integrin antibody, antigen-binding fragment thereof, multispecific molecule, conjugate, or composition administered to a subject (e.g., a mammal, such as a human) should be sufficient to affect the desired response over a reasonable time frame. According, in certain embodiments, the amount of anti-α5 integrin antibody, antigen-binding fragment thereof, multispecific molecule, conjugate, or composition described herein administered to a subject is an effective amount. In certain embodiments, the effective amount is a therapeutically effective amount.

Suitable routes of administering a presently disclosed anti-α5 integrin antibody, antigen-binding fragment thereof, multispecific molecule, conjugate, or composition are well known in the art. Depending on the circumstances, a presently disclosed anti-α5 integrin antibody, antigen-binding fragment thereof, multispecific molecule, conjugate, or composition can be applied or instilled into body cavities, absorbed through the skin or mucous membranes, ingested, inhaled, and/or introduced into circulation. In certain embodiments, a presently disclosed anti-α5 integrin antibody, antigen-binding fragment thereof, multispecific molecule, conjugate, or composition is administered through injection by intravenous, subcutaneous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. In certain embodiments, a presently disclosed anti-α5 integrin antibody, antigen-binding fragment thereof, multispecific molecule, conjugate, or composition is administered regionally via intraarterial or intravenous administration feeding the region of interest, for example, via the hepatic artery for delivery to the liver. In certain embodiments, a presently disclosed anti-α5 integrin antibody, antigen-binding fragment thereof, multispecific molecule, conjugate, or composition is administered locally via implantation of a membrane, sponge, or another appropriate material on to which the anti-α5 integrin antibody, antigen-binding fragment thereof, multispecific molecule, conjugate, or composition has been absorbed or encapsulated. Where an implantation device is used, the device is, one aspect, implanted into any suitable tissue or organ, and delivery of the anti-α5 integrin antibody, antigen-binding fragment thereof, multispecific molecule, conjugate, or composition, is, for example, via diffusion, timed-release bolus, or continuous administration. In certain embodiments, a presently disclosed anti-α5 integrin antibody, antigen-binding fragment thereof, multispecific molecule, conjugate, or composition is administered directly to exposed tissue during tumor resection or other surgical procedures.

In certain embodiments, the method further comprises administering to the subject one or more additional agents. The one or more additional agents can be administered (e.g., for combination therapy) together or separately (e.g., simultaneously, alternatively, sequentially) with the anti-α5 integrin antibody, antigen-binding fragment thereof, multispecific molecule, conjugate, or composition. In certain embodiments, the one or more additional agents comprise a therapeutic agent. Non-limiting examples of therapeutic agents include therapeutic antibodies, immunotherapies and immunotherapeutic agents, cytotoxic agents, chemotherapeutic agents, and inhibitors.

Therapeutic antibodies that can be used with the anti-α5 integrin antibody, antigen-binding fragment thereof, multispecific molecule, conjugate, or composition as described herein (e.g., for combination therapy) include, but are not limited to, an αvβ3 binding antibody (e.g., etaracizumab), an α4β1 binding antibody (e.g., natalizumab), an α4β7 binding antibody (e.g., vedolizumab), a TREM2 binding antibody (e.g., AL002), a TNFα binding antibody (e.g., adalimumab), CSF1 binding antibody (e.g., MCS110), CSF-1R binding antibody (e.g., AMG820), C1Q binding antibody (ANX005), CD40L binding antibody (e.g., ruplizumab), an FGFR antibody (e.g., bemarituzumab), IL-1β binding antibody (e.g., canakinumab, gevokizumab), IL-6 binding antibody (e.g., tocilizumab), IL-12 binding antibody (e.g., ustekinumab), and an antibody that binds type I interferons (IFN) (e.g., sifalimumab).

Immunotherapies and immunotherapeutic agents that can be used with the anti-α5 integrin antibody, antigen-binding fragment thereof, multispecific molecule, conjugate, or composition described herein (e.g., for combination therapy) include, but are not limited to, cytokines, interleukins, tumor necrosis factors, and combinations thereof. In certain embodiments, the immunotherapy includes an immunotherapeutic agent that modulates immune responses, for example, a checkpoint inhibitor or a checkpoint agonist. In certain embodiments, the immunotherapeutic agent is an antibody modulator that targets PD-1, PD-L1, PD-L2, CEACAM (eg., CEACAM-1, -3 and/or 5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In certain embodiments, the immunotherapeutic agent is an agent that increases natural killer (NK) cell activity. In certain embodiments, the immunotherapeutic agent is an agent that inhibits suppression of an immune response. In certain embodiments, the immunotherapeutic agent is an agent that inhibits suppressor cells or suppressor cell activity. In certain embodiments, the immunotherapeutic agent is an agent or therapy that inhibits Treg activity. In certain embodiments, the immunotherapeutic agent is an agent that inhibits the activity of inhibitory immune checkpoint receptors.

In certain embodiments, the immunotherapeutic agent includes a T cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, ICOS, SLAM (e.g., SLAMF7), HVEM, LIGHT, CD2, CD27, CD28, CDS, ICAM-1, LFA-1 (CD11a/CD18), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others).

Cytotoxic agents that can be used with the anti-α5 integrin antibody, antigen-binding fragment thereof, multispecific molecule, conjugate, or composition disclosed herein (e.g., for combination therapy) include a substance that inhibits or prevents a cellular function and/or causes cell death or destruction. Exemplary cytotoxic agents include, but are not limited to, radioactive isotopes (e.g., I¹³¹, I¹²⁵, Y⁹⁰, and Re¹⁸⁶); chemotherapeutic agents; and toxins of bacterial, fungal, plant or animal origin, including fragments and/or variants thereof.

Chemotherapeutic agents that can be used with the anti-α5 integrin antibody, antigen-binding fragment thereof, multispecific molecule, conjugate, or composition described herein (e.g., for combination therapy) include chemical compounds useful in the treatment of cancer. Non-limiting examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly crytophycin 1 and crytophycin 8); dolastatin; duocarmycin (including a the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1 (see, e.g., Agnew, Chem Intl. Ed. Engl., 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone mitoxantrone mopidanmol nitraerine, pentostatin; phenamet pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras and EGFR (e.g., erlotinib (Tarceva™)) that reduce cell proliferation and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON·toremifene; aromatase inhibitors that inhibit the enzyme aromatase, which regulates estrogen production in the adrenal glands, such as, for example, 4 (5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins, and pharmaceutically acceptable salts, acids or derivatives of any of the above.

Non-limiting examples of inhibitors that can be used with the anti-α5 integrin antibody, antigen-binding fragment thereof, multispecific molecule, conjugate, or composition described herein (e.g., for combination therapy) include kinase inhibitors such as FAK inhibitors (e.g., GSK2256098), MEK inhibitors (e.g., cobimetinib, rametinib, binimetinib, selumetinib), tyrosine kinase inhibitors (e.g., cabozantinib); EGFR inhibitors (e.g., erlotinib); Janus kinase (JAK)1-selective inhibitors (e.g., baricitinib, tofacitinib, upadacitinib), CSF-1R inhibitors (e.g., BLZ945); C-kit inhibitors (e.g., masitinib); and FGFR inhibitors (e.g., erdafitinib).

In certain embodiments, the anti-α5 integrin antibody, antigen-binding fragment thereof, multispecific molecule, conjugate, or composition disclosed herein can be used in combination with a PD-1 inhibitor or a PD-L1 inhibitor, e.g., an anti-PD-1 monoclonal antibody or an anti-PD-L1 monoclonal antibody, for example, nivolumab (Opdivo), pembrolizumab (Keytruda, MK-3475), atezolizumab, or avelumab.

In certain embodiments, the anti-α5 integrin antibody, antigen-binding fragment thereof, multispecific molecule, conjugate, or composition disclosed herein can be used in combination with a CTLA-4 inhibitor, e.g., an anti-CTLA-4 antibody, for example, ipilimumab (Yervoy), or with antibodies to cytokines, or with bispecific antibodies that bind to PD-L1 and CTLA-4 or PD-1 and CTLA-4, or with other anti-cancer agents.

### 4.8. Kits

The present disclosure provides kits comprising the anti-α5 integrin antibodies or antigen-binding fragments thereof, the conjugates, or the multispecific molecules disclosed herein in unit dosage form. In certain embodiments, the kit comprises a label or packaging insert including a description of the components or instructions for use in vitro, in vivo, or ex vivo, of the components therein.

In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit further comprises instructions for administering the anti-α5 integrin antibodies or antigen-binding fragments thereof, the conjugates, or the multispecific molecules disclosed herein to the subject. The instructions can generally include information about the use of the anti-α5 integrin antibodies or antigen-binding fragments thereof, the conjugates, or the multispecific molecules disclosed herein for treating an α5-associated disease, disorder, or condition. In certain embodiments, the instructions include at least one of the following: description of the therapeutic agent; dosage schedule and administration for treating an α5-associated disease, disorder, or condition; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

It is understood that modifications which do not substantially affect the activity of the various embodiments described herein are also provided within the definition of the subject matter described herein. Accordingly, the following examples are intended to illustrate but not limit the present disclosure.

### 5. EXAMPLES

The following examples are put forth so as to provide those of ordinary skill in the art with a complete disclosure and description of how to make and use the antibodies, bispecific antibodies, compositions comprising thereof, screening, and therapeutic methods of the presently disclosed subject matter, and are not intended to limit the scope of what the inventors regard as their present disclosure. It is understood that various other embodiments may be practiced, given the general description provided above.

### Example 1: Expression of Humanized Antibodies Derived from A2-M5 IgG4 Chimera

Antibody expression plasmids were constructed that encode heavy and light-chain humanized sequences with low risk (Low) and low+moderate (Low+Mod) risk amino acid changes. Correctness of the sequences was verified with Sanger sequencing and plasmid concentrations determined by measuring the absorption at a wavelength of 260 nm. Expression plasmids were combined to express heavy and light chain combinations, including V_{H} Low combined with V_{L} Low+Mod, V_{H} Low+Mod combined with V_{L} Low, and V_{H} Low+Mod combined with V_{L} Low+Mod. The expression clones were combined as described, transfected into suspension-adapted CHO K1 cells and grown in an animal-component free, serum-free medium. Supernatants were harvested by centrifugation and subsequent filtration (0.2 μm filter). The antibody was purified using MabSelect™ SuRe™ (Cytiva, Marlborough, MA). Purity was determined by analytical size exclusion chromatography with an Agilent AdvanceBio SEC column (300A 2.7 um 7.8×300 mm; Agilent Technologies, Inc., Santa Clara, CA) using PBS as running buffer at 0.8 mL/min. Yields from 250 mL culture were determined by Absorbance 280 nm for the presently disclosed humanized antibodies A2-M5-Low(H)/Low+Mod(L), A2-M5-Low+Mod(H)/Low(L), and A2-M5-Low+Mod(H)/Low+Mod(L).

Antibody expression plasmids are constructed that encode heavy and light-chain humanized sequences with low risk (Low) and low (Low) risk amino acid changes. Correctness of the sequences is verified with Sanger sequencing and plasmid concentrations determined by measuring the absorption at a wavelength of 260 nm. Expression plasmids are combined to express a combination V_{H} Low combined with V_{L} Low. The expression clones are combined as described, transfected into suspension-adapted CHO K1 cells and grown in an animal-component free, serum-free medium. Supernatants are harvested by centrifugation and subsequent filtration (0.2 μm filter). The antibody is purified using MabSelect™ SuRe™ (Cytiva, Marlborough, MA). Purity is determined by analytical size exclusion chromatography with an Agilent AdvanceBio SEC column (300A 2.7 μm 7.8×300 mm; Agilent Technologies, Inc., Santa Clara, CA) using PBS as running buffer at 0.8 mL/min. Yields from 250 mL culture are determined by Absorbance 280 nm for the presently disclosed humanized antibodies A2-M5-Low(H)/Low(L).

### Example 2: Binding Affinities of Humanized A2-M5 Antibodies

The Binding Affinities of presently disclosed humanized antibodies A2-M5-Low(H)/Low(L), A2-M5-Low(H)/Low+Mod(L), A2-M5-Low+Mod(H)/Low(L), and A2-M5-Low+Mod(H)/Low+Mod(L) are measured. Surface Plasmon Resonance (SPR) is used to measure the binding affinity of the presently disclosed humanized antibodies A2-M5-Low(H)/Low(L), A2-M5-Low(H)/Low+Mod(L), A2-M5-Low+Mod(H)/Low(L), and A2-M5-Low+Mod(H)/Low+Mod(L) to human recombinant rh-α5β1 protein using standard protocols. Briefly, rh-α5β1 protein in solution is manually printed onto a bare gold-coated sensor chip. Following the incubation, the chip is washed, blocked, re-washed, and dried prior to use. SPR measurements are performed using PlexArray HT (Plexera Bioscience, Seattle, WA), a high-throughput surface plasmon resonance imaging (SPRi) platform. Collimated light (660 nm) passes through the coupling prism, reflects off the SPR-active gold surface, and is received by the CCD camera. Buffers and samples are injected by a non-pulsatile piston pump into a flow cell that is mounted on the coupling prim. Each SPR measurement cycle contains the following steps: washing with running buffer at a constant rate to obtain a stable baseline, injection of humanized antibodies for binding to rh-α5β1 to reach equilibrium, and followed by injection of running buffer alone to allow for dissociation of humanized antibodies. All the measurements are performed at 25° C. SPR binding responses (a.u.) are recorded and plotted over time.

### Example 3: Inhibition of Fibronectin Binding to α5β1 by Humanized Antibodies

The presently disclosed humanized antibodies A2-M5-Low(H)/Low(L), A2-M5-Low(H)/Low+Mod(L), A2-M5-Low+Mod(H)/Low(L), and A2-M5-Low+Mod(H)/Low+Mod(L) are tested in a quantitative FN inhibition assay in an ELISA format. Immulon4 HBX ELISA 96-well plates are coated with FN by incubation overnight at 4° C. with 2.5 g/mL human FN (R&D Systems, Minneapolis, MN 55413, cat. no. 1918-FN) in 1×PBS (0.01 M phosphate buffer and 0.154 M NaCl, pH 7.4). Plates are then washed 3 times with Wash Buffer (1×Tris Buffered Saline containing 0.05% Tween20), blocked with 2% BSA in 1×TBS for 2 hours at room temperature (RT). Antibodies are diluted in Standard Diluent (2% BSA, 1×TBS, 0.05% Tween20) containing 0.1 μg/mL rh-α5β1-6×His tagged protein (Acro Biosystems, Newark, DE, cat. no. IT1-H52W5), to generate an 11 point 1:3 antibody dilution series ranging from 10,000 ng/ml to 0.17 ng/mL. Isotype control antibody (Control Ab; Ms IgG2a EMD Millipore Corp, Billerica, MA, cat. no. PP102) is used to normalize data across different assay runs. For the assays, 100 μL of the antibody dilution series/His-tagged-α5β1 mixture is added to the wells after the blocking solution is removed and wells are washed 3 times. Following 1 hour at RT, the wells are washed 3 times, incubated with biotinylated Anti-6×His-Tag Ab (Invitrogen, Carlsbad, CA, cat. no. MAI-21315-BTIN) at 1:1000 in Standard Diluent for 1 hour, washed 3 times, incubated for 30 minutes with poly-HRP Streptavidin (Thermo Fisher Scientific, Waltham, MA, cat. no. N200), washed 4 times, incubated with TMB substrate (Thermo Fisher Scientific, Waltham, MA, cat. no. N301) for 2-5 minutes, followed by addition of ELISA Stop Solution (Invitrogen, Carlsbad, CA, cat. no. SS04). Absorbance 450 nm is measured. Data points are normalized to the isotype Control Ab values at each concentration and reported as % Absorbance 450 nm normalized to Control Ab. Non-linear regression analysis is used to fit curves (4-parameter) to the data using GraphPad Prism version 9.0.2 (GraphPad Software, LLC, San Diego, CA).

### Example 4: Developability of Humanized A2-M5 Antibodies

The developability (including specificity, integrity, stability, and accelerated stress) are assessed and measured for the presently disclosed humanized antibodies A2-M5-Low(H)/Low(L), A2-M5-Low(H)/Low+Mod(L), A2-M5-Low+Mod(H)/Low(L), and A2-M5-Low+Mod(H)/Low+Mod(L). These assessments include mass analysis (both reduced and deglycosylated) by mass spectrometry, post-translation modification assessment by mass spectrometry, aggregation analysis by size exclusion ultra-performance chromatography (SE-UPLC), purity analysis by capillary electrophoresis sodium dodecyl sulfate (CE-SDS), charge variant analysis by capillary isoelectric focusing (cIEF) and thermostability analysis. Accelerated stress testing includes 3 cycles of freeze/thaw followed by aggregation analysis by SE-UPLC and purity analysis by CE-SDS.

### Examples 5: Immunogenicity Assessment of Humanized A2-M5 Antibodies

ProStorm® Cytokine Release Assay, designed to aid in the prediction of first infusion-related reactions, is used to assess immunogenicity of the presently disclosed humanized antibodies A2-M5-Low(H)/Low(L), A2-M5-Low(H)/Low+Mod(L), A2-M5-Low+Mod(H)/Low(L), and A2-M5-Low+Mod(H)/Low+Mod(L). This in vitro cytokine release assay is carried out on fresh human blood samples from a panel of healthy donors. A suitable volume of blood is drawn into heparin Vacutainers®. Heparinised blood samples are used fresh (<3 hours post draw) in the ProStorm® Assay. Each humanized antibody is analyzed at 4 concentrations 0.1, 1, 10, and 100 μg/mL in undiluted wholeblood along with positive and negative control conditions. After incubation, plasma is isolated and measurement of the following cytokines is made in replicates in pg/mL: IFNγ, TNFα, IL-2, IL-4, IL-6, IL-8 and IL-10 using an immunoassay.

In addition, the ProScern® Dendritic Cell-T cell (DC-T) assays are performed on the presently disclosed humanized antibodies A2-M5-Low(H)/Low(L), A2-M5-Low(H)/Low+Mod(L), A2-M5-Low+Mod(H)/Low(L), and A2-M5-Low+Mod(H)/Low+Mod(L). A panel of 20 different healthy donor PBMC samples are identified within the ProImmune Tissue Bank to reflect global HLA distribution. PBMC preparations are typed for HLA class II and stored in liquid nitrogen before use. Donor PBMC cultures are used to derive monocytes and these monocytes are induced to a semi-mature DC phenotype by culture in defined media. Following a period of culture, Dendritic cells (DCs) are incubated with the humanized antibodies in multi-well plates including un-treated control wells and wells with positive control antigens KLH and tuberculin PPD. The treated DCs are cultured in defined media to further induce maturation. These antigen-loaded DCs are harvested and re-plated for co-culture with PBMCs obtained from the same donor sample. These DC-T cell cultures are maintained in multi-well plates for an additional period of in vitro culture and T cell proliferation is determined using a CFSE staining assay. The percentage stimulation above background for each sample combination is determined by subtracting the percentage of CD4+ CFSE dim cells without antigen stimulation from the percentage of CD4+ CFSE dim cells with antigen stimulation. Statistical analysis (one-way analysis of variance) across the data set is applied.

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An anti-α5 integrin antibody or an antigen-binding fragment thereof comprising a heavy chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31.

2. The antibody or antigen-binding fragment thereof of claim 1, wherein the heavy chain variable region comprises the amino acid sequence set forth in SEQ ID NO: 31.

3. An anti-α5 integrin antibody or an antigen-binding fragment thereof comprising a heavy chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 32.

4. The antibody or antigen-binding fragment thereof of claim 3, wherein the heavy chain variable region comprises the amino acid sequence set forth in SEQ ID NO: 32.

5. An anti-α5 integrin antibody or an antigen-binding fragment thereof comprising a light chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33.

6. The antibody or antigen-binding fragment thereof of claim 5, wherein the light chain variable region comprises the amino acid sequence set forth in SEQ ID NO: 33.

7. An anti-α5 integrin antibody or an antigen-binding fragment thereof comprising a light chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 34.

8. The antibody or antigen-binding fragment thereof of claim 7, wherein the light chain variable region comprises the amino acid sequence set forth in SEQ ID NO: 34.

9. An anti-α5 integrin antibody or an antigen-binding fragment thereof comprising:
(a) a heavy chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31 or SEQ ID NO: 32; and
(b) a light chain variable region that comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33 or SEQ ID NO: 34.

10. The antibody or antigen-binding fragment thereof of claim 9, wherein
(a) the heavy chain variable region comprises the amino acid sequence set forth in SEQ ID NO: 31 or SEQ ID NO: 32; and
(b) the light chain variable region comprises the amino acid sequence set forth in SEQ ID NO: 33 or SEQ ID NO: 34.

11. The antibody or antigen-binding fragment thereof of claim 9, wherein:
(a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31; and
(b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33.

12. The antibody or antigen-binding fragment thereof of claim 11, wherein:
(a) the heavy chain variable region comprises the amino acid sequence set forth in SEQ ID NO: 31; and
(b) the light chain variable region comprises the amino acid sequence set forth in SEQ ID NO: 33.

13. The antibody or antigen-binding fragment thereof of claim 9, wherein:
(a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 31; and
(b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 34.

14. The antibody or antigen-binding fragment thereof of claim 13, wherein:
(a) the heavy chain variable region comprises the amino acid sequence set forth in SEQ ID NO: 31; and
(b) the light chain variable region comprises the amino acid sequence set forth in SEQ ID NO: 34.

15. The antibody or antigen-binding fragment thereof of claim 9, wherein:
(a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 32; and
(b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 33.

16. The antibody or antigen-binding fragment thereof of claim 15, wherein:
(a) the heavy chain variable region comprises the amino acid sequence set forth in SEQ ID NO: 32; and
(b) the light chain variable region comprises the amino acid sequence set forth in SEQ ID NO: 33.

17. The antibody or antigen-binding fragment thereof of claim 9, wherein:
(a) the heavy chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 32; and
(b) the light chain variable region comprises an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 34.

18. The antibody or antigen-binding fragment thereof of claim 17, wherein:
(a) the heavy chain variable region comprises the amino acid sequence set forth in SEQ ID NO: 32; and
(b) the light chain variable region comprises the amino acid sequence set forth in SEQ ID NO: 34.

19. The antibody or antigen-binding fragment thereof of any one of claims 1-18, wherein the antibody is a monoclonal antibody.

20. The antibody or antigen-binding fragment thereof of any one of claims 1-19, wherein the antibody is a humanized antibody.

21. The antibody or antigen-binding fragment thereof of any one of claims 1-20, wherein the antigen-binding fragment is a Fab, Fab′, F(ab′)₂, variable fragment (Fv), or single chain variable fragment (scFv).

22. The antibody or antigen-binding fragment thereof of any one of claims 1-21, which binds to α5β1 integrin.

23. The antibody or antigen-binding fragment thereof of any one of claims 1-22, which inhibits the binding of α5β1 integrin to fibronectin.

24. An antibody or an antigen-binding fragment thereof that competes with the antibody or antigen-binding fragment thereof of any one of claims 1-21 for binding to α5 integrin.

25. An antibody or an antigen-binding fragment thereof that binds to essentially the same epitope region on as the antibody or antigen-binding fragment thereof of any one of claims 1-21 for binding to α5 integrin.

26. A composition comprising the antibody or antigen-binding fragment thereof of any one of claims 1-25.

27. The composition of claim 26, which is a pharmaceutical composition comprising a pharmaceutically acceptable carrier.

28. A conjugate comprising the antibody or antigen-binding fragment thereof of any one of claims 1-25.

29. The conjugate of claim 28, wherein the antibody or antigen-binding fragment thereof is linked to a therapeutic agent, a detectable agent, or a diagnostic agent.

30. The conjugate of claim 28 or 29, which is an immunoconjugate.

31. The conjugate of claim 29, wherein the therapeutic agent is a chemotherapeutic agent, a cytotoxin, or a drug.

32. A composition comprising the conjugate of any one of claims 28-31.

33. The composition of claim 32, which is a pharmaceutical composition comprising a pharmaceutically acceptable carrier.

34. A multispecific molecule comprising the antibody or the antigen-binding fragment thereof of any one of claims 1-25, linked to a second functional moiety.

35. The multispecific molecule of claim 34, wherein the second functional moiety has a different binding specificity than said antibody or antigen binding fragment thereof.

36. A composition comprising the multispecific molecule of claim 34 or 35.

37. The composition of claim 36, which is a pharmaceutical composition comprising a pharmaceutically acceptable carrier.

38. A nucleic acid that encodes the antibody or antigen-binding fragment thereof of any one of claims 1-25.

39. The nucleic acid of claim 38, which comprises a first polynucleotide that encodes a V_{H} comprising the amino acid sequence set forth in SEQ ID NO: 31 or SEQ ID NO: 32; and/or a second polynucleotide that encodes a V_{L} comprising the amino acid sequence set forth in SEQ ID NO: 33 or SEQ ID NO: 34.

40. The nucleic acid of claim 39, wherein the first polynucleotide and the second polynucleotide are selected from the group consisting of:
(a) a first polynucleotide that encodes a V_{H} comprising the amino acid sequence set forth in SEQ ID NO: 31; and a second polynucleotide that encodes a V_{L} comprising the amino acid sequence set forth in SEQ ID NO: 33;
(b) a first polynucleotide that encodes a V_{H} comprising the amino acid sequence set forth in SEQ ID NO: 31; and a second polynucleotide that encodes a V_{L} comprising the amino acid sequence set forth in SEQ ID NO: 34;
(c) a first polynucleotide that encodes a V_{H} comprising the amino acid sequence set forth in SEQ ID NO: 32; and a second polynucleotide that encodes a V_{L} comprising the amino acid sequence set forth in SEQ ID NO: 33; and
(d) a first polynucleotide that encodes a V_{H} comprising the amino acid sequence set forth in SEQ ID NO: 32; and a second polynucleotide that encodes a V_{L} comprising the amino acid sequence set forth in SEQ ID NO: 34.

41. A vector comprising the nucleic acid of claim 40.

42. The vector of claim 41, which is an expression vector.

43. A host cell comprising the vector of claim 41 or 42.

44. A method of producing an anti-α5 integrin antibody or an antigen-binding fragment thereof, wherein the method comprises culturing the host cell of claim 43 under conditions to induce expression of the antibody or antigen-binding fragment thereof from the host cell.

45. A method for detecting α5 integrin in a whole cell or tissue, comprising: contacting a cell or tissue with the antibody or antigen-binding fragment thereof of any one of claims 1-25, wherein the antibody or antigen-binding fragment thereof comprises a detectable label; and determining the amount of the labeled antibody or antigen-binding fragment thereof bound to said cell or tissue by measuring the amount of detectable label associated with said cell or tissue, wherein the amount of bound antibody or antigen-binding fragment thereof indicates the amount of α5 integrin in said cell or tissue.

46. A method for treating an α5 integrin-associated disease, disorder or condition in a subject, comprising administering to the subject the antibody or antigen-binding fragment thereof of any one of claims 1-25, the multispecific molecule of claim 34 or 35, the conjugate of any one of claims 28-31, or the composition of any one of claims 26, 27, 32, 33, 36, and 37.

47. The method of claim 46, wherein the α5 integrin-associated disease, disorder or condition is associated with α5β1.

48. The method of claim 46 or 47, wherein the α5 integrin-associated disease, disorder or condition is a tumor.

49. The method of claim 48, wherein the tumor is a solid tumor.

50. The method of claim 48 or 49, wherein the tumor is cancer.

51. The method of claim 50, wherein the cancer is selected from the group consisting of breast cancer, bladder cancer, melanoma, prostate cancer, mesothelioma, lung cancer, brain cancer, ovarian cancer, colon cancer, testicular cancer, thyroid cancer, squamous cell carcinoma, glioblastoma, neuroblastoma, neurofibromatosis, uterine cancer, colorectal cancer, stomach cancer, and pancreatic cancer.

52. The method of claim 46 or 47, wherein the α5 integrin-associated disease, disorder or condition is associated with abnormal angiogenesis.

53. The method of claim 52, wherein the α5 integrin-associated disease, disorder or condition is an ocular disease.

54. The method of claim 53, wherein the ocular disease is selected from the group consisting of diabetic retinopathy, age-induced macular degeneration, and uveitis.

55. The method of claim 46 or 47, wherein the α5 integrin-associated disease, disorder or condition is an inflammatory disease, disorder or condition.

56. The method of claim 55, wherein the inflammatory disease, disorder, or condition is a neuroinflammatory disease, disorder or condition.

57. The method of claim 55, wherein the inflammatory disease, disorder or condition is selected from the group consisting of macrophages-mediated innate immunity diseases, multiple sclerosis, amyotrophic lateral sclerosis (ALS), and atherosclerosis.

58. A method of inhibiting abnormal angiogenesis in a subject, comprising administering to the subject the antibody or antigen-binding fragment thereof of any one of claims 1-25, the multispecific molecule of claim 34 or 35, the conjugate of any one of claims 28-31, or the composition of any one of claims 26, 27, 32, 33, 36, and 37.

59. The method of claim 58, wherein the subject suffers from a tumor.

60. The method of any one of claims 46-59, wherein the subject is human.

61. The antibody or antigen-binding fragment thereof of any one of claims 1-25, the multispecific molecule of claim 34 or 35, the conjugate of any one of claims 28-31, or the composition of any one of claims 26, 27, 32, 33, 36, and 37 for use in treating an α5 integrin-associated disease, disorder or condition in a subject or inhibiting abnormal angiogenesis in a subject.

62. The antibody or antigen-binding fragment thereof, multispecific molecule, conjugate, or composition for use of claim 61, wherein the α5 integrin-associated disease, disorder or condition is associated with α5β1.

63. The antibody or antigen-binding fragment thereof, multispecific molecule, conjugate, or composition for use of claim 61 or 62, wherein the α5 integrin-associated disease, disorder or condition is a tumor.

64. The antibody or antigen-binding fragment thereof, multispecific molecule, conjugate, or composition for use of claim 63, wherein the tumor is a solid tumor.

65. The antibody or antigen-binding fragment thereof, multispecific molecule, conjugate, or composition for use of claim 63 or 64, wherein the tumor is cancer.

66. The antibody or antigen-binding fragment thereof, multispecific molecule, conjugate, or composition for use of claim 65, wherein the cancer is selected from the group consisting of breast cancer, bladder cancer, melanoma, prostate cancer, mesothelioma, lung cancer, brain cancer, ovarian cancer, colon cancer, testicular cancer, thyroid cancer, squamous cell carcinoma, glioblastoma, neuroblastoma, neurofibromatosis, uterine cancer, colorectal cancer, stomach cancer, and pancreatic cancer.

67. The antibody or antigen-binding fragment thereof, multispecific molecule, conjugate, or composition for use of claim 61 or 62, wherein the α5 integrin-associated disease, disorder or condition is associated with abnormal angiogenesis.

68. The antibody or antigen-binding fragment thereof, multispecific molecule, conjugate, or composition for use of claim 67, wherein the α5 integrin-associated disease, disorder or condition is an ocular disease.

69. The antibody or antigen-binding fragment thereof, multispecific molecule, conjugate, or composition for use of claim 68, wherein the ocular disease is selected from the group consisting of diabetic retinopathy, age-induced macular degeneration, and uveitis.

70. The antibody or antigen-binding fragment thereof, multispecific molecule, conjugate, or composition for use of claim 61 or 62, wherein the α5 integrin-associated disease, disorder or condition is an inflammatory disease, disorder or condition.

71. The antibody or antigen-binding fragment thereof, multispecific molecule, conjugate, or composition for use of claim 70, wherein the inflammatory disease, disorder, or condition is a neuroinflammatory disease, disorder or condition.

72. The antibody or antigen-binding fragment thereof, multispecific molecule, conjugate, or composition for use of claim 70, wherein the inflammatory disease, disorder or condition is selected from the group consisting of macrophages-mediated innate immunity diseases, multiple sclerosis, amyotrophic lateral sclerosis (ALS), and atherosclerosis.

73. The antibody or antigen-binding fragment thereof, multispecific molecule, conjugate, or composition for use of any one of claims 61-72, wherein the subject suffers from a tumor.

74. The antibody or antigen-binding fragment thereof, multispecific molecule, conjugate, or composition for use of any one of claims 61-73, wherein the subject is human.
